(19) United States
(12) Patent Application Publication
(10) Pub. No.: US 2012/0115837 A1
(43) Pub. Date: May 10, 2012

(54) Solid Preparation

(75) Inventors: Wataru Hoshina, Osaka (JP); Makoto Fukuta, Osaka (JP); Shigeyuki Marunaka, Osaka (JP)

(73) Assignee: Takeda Pharmaceutical Company Limited

(21) Appl. No.: 13/138,950

(22) PCT Filed: Apr. 28, 2010
(86) PCT No.: PCT/JP2010/057923

§ 371 Date: Jan. 17, 2012

(30) Foreign Application Priority Data
Apr. 30, 2009 (JP) 2009111381
Mar. 24, 2010 (JP) 2010-068265

(51) Int. Cl. A61K 31/397; A61K 31/4245; A61P 9/12; A61K 31/444; A61K 31/496; A61K 31/416; A61K 31/4418

(52) U.S. Cl. 514/210.2; 514/381; 514/364; 514/356; 514/335; 514/253.11

(57) ABSTRACT

Provided is a solid preparation comprising (i) a compound represented by the formula (I): wherein each symbol is as defined in the specification, or a salt thereof, (ii) a sugar alcohol, and (iii) a calcium antagonist, which is superior in the dissolution property and stability.

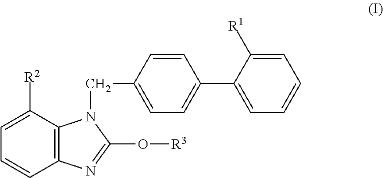

### TECHNICAL FIELD OF THE INVENTION

The present invention relates to a solid preparation showing improved dissolution property of a drug from a preparation.

### BACKGROUND OF INVENTION

Hypertension is one of the diseases most frequently found in adults. According to the 2000 circulatory disease basic research by the Ministry of Health, Labour and Welfare, the number of hypertension patients in Japan (those with systolic blood pressure of not less than 140 mmHg or diastolic blood pressure of not less than 90 mmHg, or depressor recipients) reaches about 31 million to 38 million. Hypertension is a strong risk factor for any circulatory diseases including cerebrovascular diseases and myocardial infarction. Thus, an appropriate control of the blood pressure is important for both improved prognosis of patients, and mitigation of personal and social burdens.

As therapeutic drugs for hypertension, various kinds of drugs such as hypotensive diuretics, α blockers, β blockers, angiotensin converting enzyme (ACE) inhibitors, calcium antagonists, angiotensin II receptor antagonists and the like have been developed, and many of the patients diagnosed with hypertension are under treatment with these depressors. For example, a compound represented by the following formula (I):

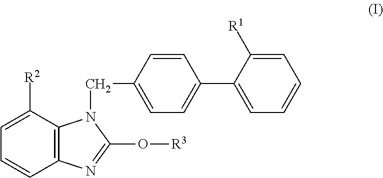

wherein R¹ is a monocyclic nitrogen-containing heterocyclic group having a deprotonatable hydrogen atom, R² is an optionally esterified carboxyl group, and R³ is an optionally substituted lower alkyl group, or a salt thereof, is known as an angiotensin II receptor antagonist showing a superior hypotensive effect and an organ-protective action. JP-B-2514282 discloses 1-(cyclohexyloxycarbonyloxy)ethyl 2-ethoxy-1-[[2′-(1H-tetrazol-5-yl)biphenyl-4-yl]methyl]benzimidazole-7-carboxylate (candesartan cilexetil) as examples of the representative medicament.

According to the J-HOME study (The Japan Home vs. Office blood pressure Measurement Evaluation study), about 40% of the hypertension patients under drug treatment have achieved a desired blood pressure (outpatient casual blood pressure of less than 140/90 mmHg), and this means some patients fail to sufficiently control the blood pressure even with the existing drug treatment. To raise the desired blood pressure achieving ratio, a more powerful hypotensive treatment is desired.

As a drug therapy exerting strong hypotensive effects, a combination therapy using plural drugs can be mentioned. For example, WO01/15674 discloses a combined use of a rennin-angiotensin inhibitor and other depressor, cholesterol-lowering drug, diuretic or the like. WO02/43807 discloses a combined use of an angiotensin II receptor antagonist and other depressor or statin. However, a combined use of plural medicaments for different timings of ingestion may adversely affect the drug compliance of patients, where a failure to control the blood pressure due to forgetfulness is feared. To more appropriately control the blood pressure, a combination preparation containing various depressors in one medicament is strongly desired in clinical practice, since it is an ideal medicament that exhibits a strong hypotensive effect and maintains drug compliance of patients.

As such combination preparation, a combination preparation containing an angiotensin II receptor antagonist and a calcium antagonist has been suggested. WO92/10097 discloses a combination preparation containing an angiotensin II receptor antagonist and other medicament such as diuretic, calcium antagonist and the like. JP-A-2006-290899 discloses a combination preparation containing an imidazolecarboxylate type angiotensin II receptor antagonist such as olmesartan medoxomil and the like and a calcium antagonist. U.S. Pat. No. 6,204,281 discloses a combination preparation containing valsartan, which is an angiotensin II receptor antagonist, and a 1,4-dihydropyridine compound such as amlodipine and the like, which is a calcium antagonist, and the like. JP-B-2930252 discloses a combination preparation containing 2-butyl-4-chloro-1-[(2′-(1H-tetrazol-5-yl)biphenyl-4-yl)methyl]imidazole-5-carboxylic acid or a pharmaceutically acceptable salt thereof, each of which is an angiotensin II receptor antagonist, and diltiazem, which is a calcium antagonist. Moreover, JP-B-3057471 discloses a combination preparation containing a benzimidazole derivative, which is an angiotensin receptor antagonist, and a diuretic or calcium antagonist.

A preparation containing a benzimidazole derivative, which is an angiotensin II receptor antagonist, and a calcium antagonist is expected to simultaneously show, in clinical practice, efficacy of the benzimidazole derivative, which is an angiotensin II receptor antagonist with a high organ-protective effect, and efficacy of the calcium antagonist with a strong hypotensive effect. Moreover, its clinical usefulness is extremely high, since the action can be enhanced and the side effects can be reduced depending on the manner of combination.

However, to secure effectiveness and safety of a pharmaceutical product, not only the effectiveness and safety of the active ingredient itself are important but also the properties of the pharmaceutical preparation, such as the drug dissolution property in the body and the like, are extremely important. For example, when dissolution of the drug from the pharmaceutical preparation is too late, the blood concentration of the drug does not reach an effective level, and the expected efficacy may not be sufficiently exhibited. On the other hand, when dissolution of the drug from the pharmaceutical preparation is too fast, the blood concentration of the drug increases rapidly, causing a high risk of side effects.

In other words, pharmaceutical products are required to ensure the certain level of the drug dissolution, in addition to the effectiveness and safety. Combination preparations are required to meet compatibility with various additives and different conditions required by each active ingredient. Thus, the development of a preparation meeting all these conditions is often difficult as compared to a preparation containing a single active ingredient. In particular, since a benzimidazole derivative, which is an angiotensin II receptor antagonist, is a poorly soluble compound, the dissolution property of the preparation may decrease due to the properties of additives and active ingredient to be combined. When the delayed release of a drug from the administered combination preparation leads to decreased drug absorbability, decreased bioavailability, i.e., decreased efficacy of active ingredient, and decreased value of the combination preparation. For a pharmaceutical preparation to be practicalized, therefore, the composition of the preparation needs to be adjusted to optimize the dissolution rate of the active ingredient in the gastrointestinal tract.

### SUMMARY OF THE INVENTION

### Problems to be Solved by the Invention

It is therefore an object of the present invention to provide a solid preparation stably containing a benzimidazole derivative having an angiotensin II receptor antagonistic action and a calcium antagonist, which is controlled to optimize dissolution property of these drugs from the preparation in the gastrointestinal tract.

### Means of Solving the Problems

The present inventors have conducted intensive studies in an attempt to solve the above-mentioned problems, and found a solid preparation comprising (i) a compound represented by the formula (I) or a salt thereof, (ii) a sugar alcohol, and (iii) a calcium antagonist, and the preparation shows appropriately controlled dissolution property in the human gastrointestinal tract. Specifically, they have taken note of excipients and found that the dissolution property of a drug from a solid preparation can be improved by using a highly water-soluble sugar alcohol, which resulted in the completion of the present invention.

Accordingly, the present invention relates to

[1] a solid preparation comprising (i) a compound represented by the formula (I):

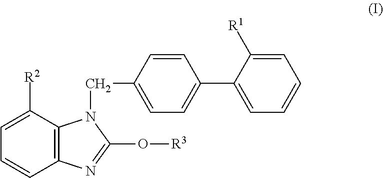

wherein R¹ is a monocyclic nitrogen-containing heterocyclic group having a deprotonatable hydrogen atom, R² is an optionally esterified carboxyl group, and R³ is an optionally substituted lower alkyl group, or a salt thereof, (ii) a sugar alcohol, and (iii) a calcium antagonist;
[1A] the solid preparation of the above-mentioned [1], wherein R² is a carboxyl group optionally esterified by a lower alkyl group having a carbon number of 1 to 4, which is optionally substituted by 1 to 3 substituents selected from a hydroxyl group, an amino group, a halogen atom, a lower alkanoyloxy group having a carbon number of 2 to 6, a lower cycloalkanoyloxy group having a carbon number of 4 to 7, a carbonyloxy group having a lower alkoxy group having a carbon number of 1 to 6, a carbonyloxy group having a lower cycloalkoxy group having a carbon number of 3 to 7, and a lower alkoxy group having a carbon number of 1 to 4; [1B] the solid preparation of the above-mentioned [1], wherein R² is a 1-(cyclohexyloxycarbonyloxy)ethoxycarbonyl group or a carboxyl group;
[2] the solid preparation of the above-mentioned [1], [1A] or [1B], wherein the compound represented by the formula (I) or a salt thereof is 1-(cyclohexyloxycarbonyloxy)ethyl 2-ethoxy-1-[[2′-(1H-tetrazol-5-yl)biphenyl-4-yl]methyl]benzimidazole-7-carboxylate, 2-ethoxy-1-[[2′-(1H-tetrazol-5-yl)biphenyl-4-yl]methyl]-1H-benzimidazole-7-carboxylic acid, 2-ethoxy-1-[[2′-(4,5-dihydro-5-oxo-1,2,4-oxadiazol-3-yl)biphenyl-4-yl]methyl]-1H-benzimidazole-7-carboxylic acid or a salt thereof;
[3] the solid preparation of the above-mentioned [1], [2], [1A] or [1B], wherein the compound represented by the formula (I) or a salt thereof is 1-(cyclohexyloxycarbonyloxy)ethyl 2-ethoxy-1-[[2′-(1H-tetrazol-5-yl)biphenyl-4-yl]methyl]benzimidazole-7-carboxylate or a salt thereof;
[4] the solid preparation of the above-mentioned [1], [2], [1A] or [1B], wherein the compound represented by the formula (I) or a salt thereof is 2-ethoxy-1-[[2′-(4,5-dihydro-5-oxo-1,2,4-oxadiazol-3-yl)biphenyl-4-yl]methyl]-1H-benzimidazole-7-carboxylic acid or a salt thereof;
[5] the solid preparation of the above-mentioned [1], [2], [3], [4], [1A] or [1B], wherein the sugar alcohol is mannitol, sorbitol or erythritol;
[6] the solid preparation of the above-mentioned [1], [2], [3], [4], [5], [1A] or [1B], wherein the sugar alcohol is mannitol;
[7] the solid preparation of the above-mentioned [1], [2], [3], [4], [5], [6], [1A] or [1B], wherein the calcium antagonist is azelnidipine, amlodipine, aranidipine, efonidipine, cilnidipine, nicardipine, nisoldipine, nitrendipine, nifedipine, nilvadipine, barnidipine, felodipine, benidipine, manidipine or a salt thereof;
[8] the solid preparation of the above-mentioned [1], [2], [3], [4], [5], [6], [7], [1A] or [1B], wherein the calcium antagonist is amlodipine or a salt thereof;
[9] the solid preparation of the above-mentioned [1], [2], [3], [4], [5], [6], [7], [8], [1A] or [1B], further comprising a polyethylene glycol;
[10] the solid preparation of the above-mentioned [9], wherein the polyethylene glycol has a molecular weight of 1,000 to 10,000;
[11] a solid preparation comprising (i) 1-(cyclohexyloxycarbonyloxy)ethyl 2-ethoxy-1-[[2′-(1H-tetrazol-5-yl)biphenyl-4-yl]methyl]benzimidazole-7-carboxylate or a salt thereof, (ii) mannitol, and (iii) amlodipine or a salt thereof;
[11A] the solid preparation of the above-mentioned [11], further comprising a polyethylene glycol;
[11B] the solid preparation of the above-mentioned [11A], wherein the polyethylene glycol has a molecular weight of 1,000 to 10,000 (preferably 3,000 to 10,000);
[11C] the solid preparation of the above-mentioned [11B], wherein the content of the polyethylene glycol is 1 to 5 wt %;
[11D] the solid preparation of the above-mentioned [11B], wherein the content of the polyethylene glycol is 1 to 3 wt %;
[12] a solid preparation comprising (i) 2-ethoxy-1-[[2′-(4,5-dihydro-5-oxo-1,2,4-oxadiazol-3-yl)biphenyl-4-yl]methyl]-1H-benzimidazole-7-carboxylic acid or a salt thereof, (ii) mannitol, and (iii) amlodipine or a salt thereof;
[12A] the solid preparation of the above-mentioned [12], further comprising polyethylene glycol;
[12B] the solid preparation of the above-mentioned [12A], wherein the polyethylene glycol has a molecular weight of 1,000 to 10,000 (preferably 3,000 to 10,000);
[12C] the solid preparation of the above-mentioned [12B], wherein the content of the polyethylene glycol is 1 to 5 wt %;
[12D] the solid preparation of the above-mentioned [12B], wherein the content of the polyethylene glycol is 1 to 3 wt %;
[13] the solid preparation of the above-mentioned [1], [2], [3], [4], [5], [6], [7], [8], [9], [10], [11], [12], [1A], [1B], [11A], [11B], [11C], [11D], [12A], [12B], [12C] or [12D], which is a tablet;
[14] the solid preparation of the above-mentioned [1], [2], [3], [4], [5], [6], [7], [8], [9], [10], [11], [12], [1A], [13], [11A], [11B], [11C], [11D], [12A], [12B], [12C] or [12D], which is a single-layer tablet;
[15] the solid preparation of the above-mentioned [1], [2], [3], [4], [5], [6], [7], [8], [9], [10], [11], [12], [13], [14], [1A], [1B], [11A], [11B], [11C], [11D], [12A], [12B], [12C] or [12D], which is a prophylactic or therapeutic drug for hypertension, cardiac failure, diabetic nephropathy or arteriosclerosis;
[16] the solid preparation of the above-mentioned [1], [2], [3], [4], [5], [6], [7], [8], [9], [10], [11], [12], [13], [14], [1A], [1B], [11A], [11B], [11C], [11D], [12A], [12B], [12C] or [12D], which is a prophylactic or therapeutic drug for hypertension;
and the like.

### Effect of the Invention

According to the present invention, a solid preparation wherein the dissolution of a compound represented by the above-mentioned formula (I) or a salt thereof, and a calcium antagonist from the preparation in the gastrointestinal tract is appropriately controlled, and the stability thereof in the preparation is finely maintained can be obtained. That is, the solid preparation of the present invention is superior in the dissolution property of a compound represented by the above-mentioned formula (I) or a salt thereof and a calcium antagonist from a preparation and the stability thereof.

### DETAILED DESCRIPTION OF THE INVENTION

The solid preparation of the present invention is explained in detail in the following.

The solid preparation of the present invention is a solid preparation comprising (i) a compound represented by the following formula (I):

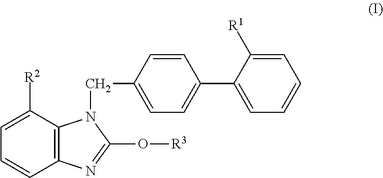

wherein R¹ is a monocyclic nitrogen-containing heterocyclic group having a deprotonatable hydrogen atom, R² is an optionally esterified carboxyl group, and R³ is an optionally substituted lower alkyl group, or a salt thereof, (ii) sugar alcohol, and (iii) a calcium antagonist (hereinafter to be also referred to as the solid preparation of the present invention).

In the above-mentioned formula (I), as the monocyclic nitrogen-containing heterocyclic group having a deprotonatable hydrogen atom for R¹, for example, a tetrazolyl group and a group represented by the formula

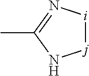

wherein i is —O— or —S— and j is >C═O, >C═S or >S(O)ₘ wherein m is 0, 1 or 2,
and the like can be mentioned. As a preferable group, a tetrazolyl group, 4,5-dihydro-5-oxo-1,2,4-oxadiazol-3-yl group and the like can be mentioned.

4,5-Dihydro-5-oxo-1,2,4-oxadiazol-3-yl group contains 3 tautomers (a′, b′ and c′) represented by the formulas:

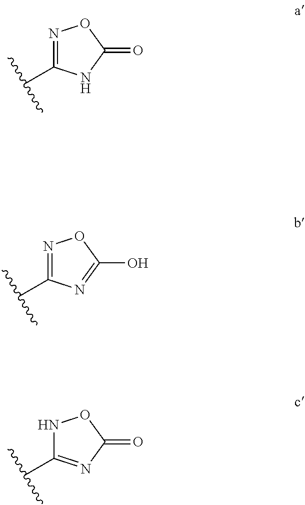

and the 4,5-dihydro-5-oxo-1,2,4-oxadiazol-3-yl group includes all of the above-mentioned a′, b′ and c′.

In the above-mentioned formula (I), examples of the optionally esterified carboxyl group for R² include a carboxyl group optionally esterified by a lower alkyl group having a carbon number of 1 to 4. The aforementioned lower alkyl group is optionally substituted by 1 to 5 (preferably 1 to 3) substituent(s) selected from the group consisting of a hydroxyl group, an amino group, a halogen atom, a lower alkanoyloxy group having a carbon number of 2 to 6 (e.g., acetyloxy group, pivaloyloxy group and the like), a lower cycloalkanoyloxy group having a carbon number of 4 to 7, a carbonyloxy group having a lower alkoxy group having a carbon number of 1 to 6 (e.g., methoxycarbonyloxy group, ethoxycarbonyloxy group and the like), a carbonyloxy group having a lower cycloalkoxy group having a carbon number of 3 to 7 (e.g., cyclohexyloxycarbonyloxy group and the like), and a lower alkoxy group having a carbon number of 1 to 4. As a preferable group, a 1-(cyclohexyloxycarbonyloxy)ethoxycarbonyl group, a carboxyl group and the like can be mentioned.

In the above-mentioned formula (I), as the optionally substituted lower alkyl group for R³, a lower alkyl group having a carbon number of 1 to 5, which is optionally substituted by 1 to 5 (preferably 1 to 3) substituent(s) selected from the group consisting of a hydroxyl group, an amino group, a halogen atom and a lower alkoxy group having a carbon number of 1 to 4, can be mentioned. Preferred is a lower alkyl group having a carbon number of 2 to 3, and particularly preferred is an ethyl group.

The salt of the compound represented by the above-identified formula (I) only needs to be a pharmaceutically acceptable salt and, for example, a salt with an inorganic base, a salt with an organic base, a salt with an inorganic acid, a salt with an organic acid, a salt with a basic or acidic amino acid and the like of a compound represented by the formula (I) can be mentioned. Preferable examples of the salt with an inorganic base include alkali metal salts such as sodium salt, potassium salt and the like; alkaline earth metal salts such as calcium salt, magnesium salt and the like; aluminum salt, ammonium salt and the like. Preferable examples of the salt with an organic base include salts with trimethylamine, triethylamine, pyridine, picoline, ethanolamine, diethanolamine, triethanolamine, dicyclohexylamine, N,N′-dibenzylethylenediamine and the like. Preferable examples of the salt with an inorganic acid include salts with hydrochloric acid, hydrobromic acid, nitric acid, sulfuric acid, phosphoric acid and the like. Preferable examples of the salt with an organic acid include salts with formic acid, acetic acid, trifluoroacetic acid, fumaric acid, oxalic acid, tartaric acid, maleic acid, citric acid, succinic acid, malic acid, methanesulfonic acid, benzenesulfonic acid, p-toluenesulfonic acid and the like. Preferable examples of the salt with a basic amino acid include salts with arginine, lysine, ornithine and the like. Preferable examples of the salt with an acidic amino acid include salts with aspartic acid, glutamic acid and the like.

The compound represented by the above-mentioned formula (I) or a salt thereof may be a hydrate or a non-hydrate, and a solvate or a nonsolvate.

Moreover, the compound represented by the above-mentioned formula (I) or a salt thereof is preferably crystalline, and the melting point is 100 to 250° C., preferably 120 to 200° C., particularly 130 to 180° C.

As the solid preparation of the present invention, a compound represented by the above-mentioned formula (I) or a salt thereof is used. Preferable examples of the compound or a salt thereof include 1-(cyclohexyloxycarbonyloxy)ethyl 2-ethoxy-1-[[2′-(1H-tetrazol-5-yl)biphenyl-4-yl]methyl]benzimidazole-7-carboxylate, 2-ethoxy-1-[[2′-(1H-tetrazol-5-yl)biphenyl-4-yl]methyl]-1H-benzimidazole-7-carboxylic acid, pivaloyloxymethyl 2-ethoxy-1-[[2′-(1H-tetrazol-5-yl)biphenyl-4-yl]methyl]-1H-benzimidazole-7-carboxylate, 2-ethoxy-1-[[2′-(4,5-dihydro-5-oxo-1,2,4-oxadiazol-3-yl)biphenyl-4-yl]methyl]-1H-benzimidazole-7-carboxylic acid, and salts thereof. Among these, 1-(cyclohexyloxycarbonyloxy)ethyl 2-ethoxy-1-[[2′-(1H-tetrazol-5-yl)biphenyl-4-yl]methyl]benzimidazole-7-carboxylate and a salt thereof, 2-ethoxy-1-[[2′-(4,5-dihydro-5-oxo-1,2,4-oxadiazol-3-yl)biphenyl-4-yl]methyl]-1H-benzimidazole-7-carboxylic acid and a salt thereof are particularly preferable.

In the present invention, the compound represented by the above-mentioned formula (I) or a salt thereof is contained in the solid preparation of the present invention in a proportion of 0.1 to 60 wt %, preferably 1 to 40 wt %, more preferably 3 to 30 wt % based on a free form.

As sugar alcohol to be used in the present invention, any sugar alcohols can be used as long as it simultaneously establishes the stability of the compound represented by the above-mentioned formula (I) or a salt thereof in a preparation, and the dissolution property thereof from a preparation and can be applied to pharmaceutical products. Examples of the sugar alcohol to be used in the present invention include monosaccharide sugar alcohols such as tetritol (e.g., erythritol, D-threitol, L-threitol and the like), pentitol (e.g., D-arabinitol, xylitol and the like), hexitol (e.g., D-iditol, galactitol (dulcitol), D-glucitol (sorbitol), mannitol and the like), cyclitol (e.g., inositol and the like) and the like; disaccharide sugar alcohols such as maltitol, lactitol, reduced paratinose (isomalt) and the like; oligosaccharide sugar alcohols such as pentaerythritol, hydrogenated maltose starch syrup and the like; and the like. Among these, monosaccharide sugar alcohols are preferable. More preferable are mannitol, sorbitol and erythritol. Particularly, mannitol is preferable, and D-mannitol is especially preferable. The sugar alcohol may be used alone or two or more kinds thereof may be used in combination. In addition, the sugar alcohol simultaneously achieves the stability of a calcium antagonist in a preparation and dissolution property thereof from the preparation.

In the present invention, the sugar alcohol is contained in the solid preparation of the present invention in a proportion of 15 to 85 wt %, preferably 20 to 80 wt %, more preferably 25 to 75 wt %.

Examples of the calcium antagonist to be used in the present invention include dihydropyridine compounds such as azelnidipine, amlodipine, aranidipine, efonidipine, cilnidipine, nicardipine, nisoldipine, nitrendipine, nifedipine, nilvadipine, barnidipine, felodipine, benidipine, manidipine and the like; benzodiazepine compounds such as diltiazem and the like; and the like. The calcium antagonist to be used in the present invention also includes salts of the compounds recited as the aforementioned calcium antagonist.

As the calcium antagonist to be used in the present invention, dihydropyridine compounds, particularly amlodipine or a salt thereof is preferable. Among these, a salt of amlodipine is more preferable, and amlodipine besylate is particularly preferable.

In the present invention, a calcium antagonist is contained in the solid preparation of the present invention in a proportion of generally 0.05 to 60 wt %, preferably 0.1 to 40 wt %, more preferably 0.5 to 20 wt %, based on a free form. Specifically, for example, amlodipine is contained in a proportion of generally 0.05 to 60 wt %, preferably 0.1 to 40 wt %, more preferably 0.5 to 20 wt %, based on a free form.

The solid preparation of the present invention may further contain an alkylene oxide polymer. Examples of the alkylene oxide polymer include a polymer of ethylene oxide, propylene oxide, trimethylene oxide, tetrahydrofuran or the like (preferably, ethylene oxide). The molecular weight of the alkylene oxide polymer is preferably 1,000 to 10,000, more preferably 3,000 to 10,000. The alkylene oxide polymer may be a copolymer of alkylene oxide, and examples of the copolymer of alkylene oxide include copolymer of two or more kinds of the above-mentioned alkylene oxide, which has a molecular weight of 1,000 to 10,000 (preferably 3,000 to 10,000).

The alkylene oxide polymer may be used alone or two or more kinds thereof may be used in combination.

As the alkylene oxide polymer to be used in the present invention, polyethylene glycol is preferable, polyethylene glycol having a molecular weight of 1,000 to 10,000 is more preferable, and polyethylene glycol (e.g., polyethylene glycol 4000, polyethylene glycol 6000, polyethylene glycol 10000) having a molecular weight of 3,000 to 10,000 is particularly preferable.

In the present invention, the alkylene oxide polymer is contained in the solid preparation of the present invention in a proportion of preferably 1 to 5 wt %, more preferably 1 to 3 wt %.

A preferable embodiment of the solid preparation of the present invention includes

a solid preparation wherein the compound represented by the above-mentioned formula (I) or a salt thereof is 1-(cyclohexyloxycarbonyloxy)ethyl 2-ethoxy-1-[[2′-(1H-tetrazol-5-yl)biphenyl-4-yl]methyl]benzimidazole-7-carboxylate (hereinafter sometimes referred to as “compound A”) or a salt thereof, and the calcium antagonist is amlodipine besylate; a solid preparation wherein the compound represented by the above-mentioned formula (I) or a salt thereof is 2-ethoxy-1-[[2′-(4,5-dihydro-5-oxo-1,2,4-oxadiazol-3-yl)biphenyl-4-yl]methyl]-1H-benzimidazole-7-carboxylic acid (hereinafter sometimes referred to as “compound B”) or a salt thereof and the calcium antagonist is amlodipine besylate;
and the like.

As the solid preparation of the present invention, for example, a solid preparation suitable for oral administration such as tablet, granule, fine granule, capsule, pill and the like can be mentioned, with preference given to tablet and more preference given to single-layer tablet. When the solid preparation of the present invention is a tablet, the form of the tablet is any of round, oval, oblong and the like. While the size of the tablet varies depending on the form of the tablet (round, caplet, oblong etc.), any size is possible as long as patients can take the tablet with ease. When the solid preparation of the present invention is a single-layer tablet, it is easily taken due to its small tablet size.

The embodiment of the solid preparation of the present invention includes

### (1) One Group Granulated Preparation

a solid preparation obtained by granulating a mixture comprising a compound represented by the formula (I) or a salt thereof, sugar alcohol, and a calcium antagonist (preferably, a compound represented by the formula (I) or a salt thereof, sugar alcohol, a calcium antagonist, and a polyethylene glycol), and compression molding the obtained granulated material (e.g., one group granulated single-layer tablet);

### (2) Two Group Granulated Preparation

(a) a solid preparation obtained by mixing and compression molding the following first part and the second part obtained by individual granulation (e.g., two group granulated single-layer tablet);
(b) a solid preparation obtained by compression molding (without mixing) the following first part and the second part obtained by individual granulation (e.g., multi-layer tablet);
(c) a solid preparation obtained by coating one of the following first part and the second part obtained by individual granulation with the other (e.g., dry coated tablet);

first part: a part comprising a compound represented by the formula (I) or a salt thereof (preferably, a compound represented by the formula (I) or a salt thereof, and a polyethylene glycol)

second part: a part comprising sugar alcohol and a calcium antagonist

and the like.

The preferable embodiment of the solid preparation of the present invention is one group granulated preparation (e.g., one group granulated single-layer tablet).

The solid preparation of the present invention may contain additives conventionally used in the pharmaceutical field. Examples of the additive include excipient, disintegrant, binder, lubricant, pH adjuster, colorant, surfactant, stabilizer, acidulant, flavor, glidant and the like. These additives are used in amounts conventionally employed in the pharmaceutical field. In addition, these additives may be used in a mixture of two or more kinds thereof at an appropriate ratio.

Examples of the excipient include starches such as corn starch, potato starch, wheat starch, rice starch, partly pregelatinized starch, pregelatinized starch, porous starch and the like, saccharides such as lactose, fructose, glucose, sucrose and the like, anhydrous calcium phosphate, crystalline cellulose, microcrystalline cellulose, Glycyrrhiza uralensis, sodium hydrogen carbonate, calcium phosphate, calcium sulfate, calcium carbonate, precipitated calcium carbonate, calcium silicate and the like.

Examples of the disintegrant include amino acid, starch, corn starch, carboxymethylcellulose, calcium carboxymethylcellulose, sodium carboxymethyl starch, carmellose sodium, carmellose calcium, croscarmellose sodium, crospovidone, low-substituted hydroxypropylcellulose, hydroxypropylstarch and the like.

In the present invention, a disintegrant is contained in a proportion of preferably 0.1 to 30 wt %, more preferably 1 to 10 wt %, of the solid preparation of the present invention.

Examples of the binder include crystalline cellulose (e.g., microcrystalline cellulose), hydroxypropylcellulose, hypromellose, polyvinylpyrrolidone, gelatin, starch, gum arabic powder, tragacanth, carboxymethylcellulose, sodium alginate, pullulan, glycerol and the like.

In the present invention, a binder is contained in a proportion of preferably 0.1 to 40 wt %, more preferably 1 to 10 wt %, of the solid preparation of the present invention.

Examples of the lubricant include magnesium stearate, stearic acid, calcium stearate, talc (purified talc), sucrose esters of fatty acids, sodium stearyl fumarate and the like.

Examples of the pH adjuster include citric acid and a salt thereof, phosphoric acid and a salt thereof, carbonic acid and a salt thereof, tartaric acid and a salt thereof, fumaric acid and a salt thereof, acetic acid and a salt thereof, amino acid and a salt thereof and the like.

Examples of the colorant include food colors such as Food Color Yellow No. 5, Food Color Red No. 2, Food Color Blue No. 2 and the like, food lake colors such as Food Yellow No. 4 Aluminum Lake and the like, iron oxide pigments such as red ferric oxide (colcothar), yellow ferric oxide, triiron tetraoxide (iron oxide black) and the like; and so on.

Examples of the surfactant include sodium lauryl sulfate, polysorbate 80, polyoxyethylene(160)polyoxypropylene(30)glycol and the like.

Examples of the stabilizer include tocopherol, tetrasodium edetate, nicotinamide, cyclodextrins and the like.

Examples of the acidulant include ascorbic acid, citric acid, tartaric acid, malic acid and the like.

Examples of the flavor include menthol, peppermint oil, lemon oil, vanillin and the like.

Examples of the glidant include light anhydrous silicic acid, hydrated silicon dioxide and the like.

The solid preparation of the present invention can also be processed into a film-coated preparation by applying a film coating such as a coating base, a coating additive and the like. Examples of the film-coated preparation include a sugar-coated preparation, sustained-release preparation, enteric preparation and the like.

Preferable examples of the coating base include a sugar coating base, a water-soluble film coating base, an enteric film coating base, a sustained-release film coating base and the like.

As the sugar coating base, sucrose is used. Furthermore, one or more kinds selected from talc, precipitated calcium carbonate, gelatin, gum arabic, pullulan, Carnauba wax and the like may be used in combination.

Examples of the water-soluble film coating base include cellulose polymers such as hydroxypropylcellulose [e.g., NISSO HPC (grades: L, SL, SL-T, SSL) (trade name); Nippon Soda Co., Ltd.], hypromellose [e.g., TC-5 (grades: MW, E, EW, R, RW) (trade name); Shin-Etsu Chemical Co., Ltd.], hydroxyethylcellulose, methylhydroxyethylcellulose and the like, synthetic polymers such as polyvinylacetal diethylaminoacetate, aminoalkylmethacrylate copolymer E [Eudragit E (trade name); manufactured by ROHM AND HAAS JAPAN KK.], polyvinylpyrrolidone and the like, polysaccharides such as pullulan and the like; and so on.

Examples of the enteric film coating base include cellulose polymers such as hypromellose phthalic acid ester, hydroxypropylmethylcellulose acetate succinate, carboxymethylethylcellulose, cellulose acetate phthalate and the like, acrylic acid polymers such as methacrylic acid copolymer L [Eudragit L (trade name)], methacrylic acid copolymer LD [Eudragit L-30D55 (trade name); manufactured by ROHM AND HAAS JAPAN KK.], methacrylic acid copolymer S [Eudragit S (trade name); manufactured by ROHM AND HAAS JAPAN KK.] and the like, natural polymers such as shellac and the like; and so on.

Examples of the sustained-release film coating base include cellulose polymers such as ethylcellulose and the like, acrylic acid polymers such as aminoalkylmethacrylate copolymer RS [Eudragit RS (trade name); manufactured by ROHM AND HAAS JAPAN KK.], ethyl acrylate-methyl methacrylate copolymer suspension [Eudragit NE (trade name); manufactured by ROHM AND HAAS JAPAN KK.] and the like; and so on.

Preferable examples of the coating additives include light protecting agents such as titanium dioxide and the like, glidants such as talc and the like, colorants such as red ferric oxide, yellow ferric oxide and the like, plasticizers such as polyethylene glycol [e.g., macrogol 6000 (trade name); manufactured by Sanyo Chemical Industries, Ltd.], triethyl citrate, castor oil, polysorbates and the like, organic acids such as citric acid, tartaric acid, malic acid, ascorbic acid and the like; and so on.

The solid preparation of the present invention can be produced by a method known per se (e.g., the method described in the Japanese Pharmacopoeia 15th Edition, General Principles).

Examples of the method include operations such as mixing, kneading, granulation, compression molding, film coating and the like, and an appropriate combination of these operations.

Mixing is performed using, for example, a mixer such as a horizontal cylindrical mixer, a V-type mixer, a tumbler mixer and the like, and kneading is performed using a rotary vessel-type kneader such as a ball mill and the like, a fixed vessel-type kneader such as a screw-type kneader, a Henschel mixer and the like, a rolling kneader such as a rolling mill, a taper rolling mill and the like, and the like. Granulation is performed by a stirring granulation method using a high-speed stirring granulator, a rolling granulation method using a container of a pan type, conical drum type, multi-stage conical drum type, stirring blade-equipped drum type, vibration type or the like, a fluidized bed granulation and drying method, a spray drying granulation method, an extrusion-granulation method, a method using a granulator such as a Roller-compactor and the like, and the like.

Compression molding is performed, for example, by molding by an extrusion molding machine, or tableting using a single punch tableting machine, a rotary tableting machine and the like.

When compression molding is performed using a single stroke tableting machine, a rotary tableting machine and the like, it is generally preferable to employ a tableting pressure of 1 to 35 kN/cm² (preferably 5 to 35 kN/cm²), and further, a taper cutting die to prevent capping.

Film coating is performed, for example, by using a pan coating machine of a horizontal type, an inclination type and the like, a fluid coating machine of a horizontal rotating-disc type, an inclined plate type and the like, a fluid coating machine of a fluidized bed type, a spouted bed type, a tumbling fluidized bed type and the like, and the like.

The solid preparation of the present invention can be produced, for example, by the following production steps.

(1) When the solid preparation of the present invention is a one group granulated preparation

A mixture comprising a compound represented by the above-mentioned formula (I) or a salt thereof, sugar alcohol, and a calcium antagonist (preferably, a compound represented by the formula (I) or a salt thereof, sugar alcohol, a calcium antagonist, and a polyethylene glycol) is granulated, and the obtained granulated material is compression molded to give the solid preparation of the present invention (one group granulated single-layer tablet).

Specifically, a compound represented by the above-mentioned formula (I) or a salt thereof, a calcium antagonist and an additive such as excipient, sugar alcohol (e.g., D-mannitol) and the like are mixed, and the mixture is granulated while spraying a liquid obtained by dispersing or dissolving polyethylene glycol and an additive such as a binder and the like in a solvent (e.g., water).

An additive such as disintegrant, lubricant and the like is added to the thus-obtained granulated material and, after mixing, the mixture is compression molded to give a tablet. The tablet is coated with a film solution containing a coating base and the like, whereby the solid preparation of the present invention (one group granulated single-layer tablet) is produced.

(2) When the solid preparation of the present invention is a two group granulated preparation
(a) two group granulated single-layer tablet;

A compound represented by the above-mentioned formula (I) or a salt thereof and an additive such as excipient and the like are mixed, and the mixture is granulated while spraying a liquid obtained by dispersing or dissolving polyethylene glycol and an additive such as a binder and the like in a solvent (e.g., water).

On the other hand, a calcium antagonist and an additive such as excipient, sugar alcohol (e.g., D-mannitol) and the like are mixed, and the mixture is granulated while spraying a liquid obtained by dispersing or dissolving an additive such as a binder and the like in a solvent (e.g., water).

An additive such as disintegrant, lubricant and the like is added to the thus-obtained granulated material containing a compound represented by the above-mentioned formula (I) or a salt thereof and the thus-obtained granulated material containing the calcium antagonist and, after mixing, the mixtures are compression molded to give the solid preparation of the present invention (two group granulated single-layer tablet).

(b) multi-layer tablet;

A compound represented by the above-mentioned formula (I) or a salt thereof and an additive such as excipient and the like are mixed, and the mixture is granulated while spraying a liquid obtained by dispersing or dissolving polyethylene glycol and an additive such as a binder and the like in a solvent (e.g., water). An additive such as disintegrant, lubricant and the like is added to the thus-obtained granulated material to give a mixed granule.

On the other hand, a calcium antagonist and an additive such as excipient, sugar alcohol (e.g., D-mannitol) and the like are mixed, and the mixture is granulated while spraying a liquid obtained by dispersing or dissolving an additive such as a binder and the like in a solvent (e.g., water). An additive such as disintegrant, lubricant and the like is added to the thus-obtained granulated material to give a mixed granule.

The thus-obtained mixed granule containing a compound represented by the above-mentioned formula (I) or a salt thereof and the thus-obtained mixed granule containing the calcium antagonist are laid on top of each other and compression molded to give the solid preparation of the present invention (multi-layer tablet).

(c) dry coated tablet;

A calcium antagonist and an additive such as excipient, sugar alcohol (e.g., D-mannitol) and the like are mixed, and the mixture is granulated while spraying a liquid obtained by dispersing or dissolving an additive such as a binder and the like in a solvent (e.g., water). An additive such as disintegrant, lubricant and the like is added to the obtained granulated material and, after mixing, the mixture is compression molded to give a tablet. The tablet is coated with a film solution containing a coating base and the like to give an inner core tablet.

On the other hand, a compound represented by the above-mentioned formula (I) or a salt thereof and an additive such as excipient and the like are mixed, and the mixture is granulated while spraying a liquid obtained by dispersing or dissolving polyethylene glycol and an additive such as a binder and the like in a solvent (e.g., water). An additive such as disintegrant, lubricant and the like is added to the obtained granulated material to give a mixed granule.

The mixed granule is added as an outer layer to the above-mentioned inner core tablet and they are compression molded to give the solid preparation of the present invention (dry coated tablet).

When the solid preparation of the present invention is granules or fine granules, they can be produced by a method similar to the above-mentioned methods.

When the solid preparation of the present invention is a capsule, it can be produced by filling the above-mentioned granules or fine granules in a capsule containing gelatin, hypromellose and the like. Furthermore, a hard capsule can be produced by filling a compound represented by the above-mentioned formula (I) or a salt thereof and a calcium antagonist together with a sugar alcohol and other excipient etc. in a capsule containing gelatin, hypromellose and the like. In addition, a soft capsule can be produced by encapsulation molding a compound represented by the above-mentioned formula (I) or a salt thereof and a calcium antagonist into a given shape with a base containing gelatin and a plasticizer such as glycerol and the like.

The solid preparation of the present invention may have a punch mark for identification or print on the surface. In addition, it may have a score line for partition.

The solid preparation of the present invention is low toxic and can be safely administered orally or parenterally as a medicament for a mammal (e.g., human, monkey, cat, swine, horse, bovine, mouse, rat, guinea pig, dog, rabbit and the like).

A compound represented by the formula (I) or a salt thereof has a strong angiotensin II receptor antagonistic action, and therefore, the solid preparation of the present invention is useful as a prophylactic or therapeutic drug for (1) a disease developed (or promoted to be developed) by coarctation or growth of blood vessel or an organ disorder expressed via angiotensin II receptor, (2) a disease developed (or promoted to be developed) by the presence of angiotensin II, or (3) a disease developed (or promoted to be developed) by a factor induced by the presence of angiotensin II in the aforementioned mammals.

Examples of the diseases of the above-mentioned (1) to (3) include hypertension, blood pressure circadian rhythm abnormality, heart diseases (e.g., cardiac hypertrophy, acute heart failure, chronic heart failure including cardiac failure, failure of expansion, cardiac myopathy, angina pectoris, myocarditis, atrial fibrillation, arrhythmia, tachycardia, cardiac infraction etc.), cerebrovascular disorders (e.g., asymptomatic cerebrovascular disorder, transient cerebral ischemia, apoplexy, cerebrovascular dementia, hypertensive encephalopathy, cerebral infarction etc.), cerebral edema, cerebral circulatory disorder, recurrence and sequela of cerebrovascular disorders (e.g., neurotic symptom, psychic symptom, subjective symptom, disorder in daily living activities etc.), ischemic peripheral circulation disorder, myocardial ischemia, venous insufficiency, progression of cardiac insufficiency after myocardial infarction, renal diseases (e.g., nephritis, glomerulonephritis, glomerulosclerosis, renal failure, thrombotic vasculopathy, diabetic nephropathy, complication of dialysis, organ damage including nephropathy by radiation irradiation etc.), arteriosclerosis including atherosclerosis (e.g., aneurysm, coronary sclerosis, cerebral arteriosclerosis, peripheral arterial sclerosis etc.), vascular hypertrophy, vascular hypertrophy or obliteration and organ damages after intervention (e.g., percutaneous transluminal coronary angioplasty, stenting, coronary angioscopy, intravascular ultrasound, dounce thrombolytic therapy etc.), vascular re-obliteration and restenosis after bypass, polycythemia, hypertension, organ damage and vascular hypertrophy after transplantation, rejection after transplantation, ocular diseases (e.g., glaucoma, ocular hypertension etc.), thrombosis, multiple organ disorder, endothelial dysfunction, hypertensive tinnitus, other cardiovascular diseases (e.g., deep vein thrombosis, obstructive peripheral circulatory disorder, arteriosclerosis obliterans, thromboangiitis obliterans, ischemic cerebral circulatory disorder, Raynaud's disease, Berger disease etc.), metabolic and/or nutritional disorders (e.g., obesity, hyperlipidemia, hypercholesterolemia, hyperuricacidemia, hyperkalemia, hypernatremia etc.), neurodegenerative diseases (e.g., Alzheimer's disease, Parkinson's disease, amyotrophic lateral sclerosis, AIDS encephalopathy etc.), central nervous system disorders (e.g., damages such as cerebral hemorrhage and cerebral infarction, and sequela and complication thereof, head injury, spinal injury, cerebral edema, senile dementia, sensory malfunction, sensory functional disorder, autonomic nervous system disorder, autonomic nervous system malfunction, multiple sclerosis etc.), dementia, defects of memory, disorder of consciousness, amnesia, anxiety symptom, catatonic symptom, discomfort mental state, psychopathies (e.g., depression, epilepsy, alcoholism etc.), inflammatory diseases (e.g., arthritis such as rheumatoid arthritis, osteoarthritis, rheumatoid myelitis, periostitis etc.; inflammation after operation or injury; remission of swelling; pharyngitis; cystitis; pneumonia; atopic dermatitis; inflammatory intestinal diseases such as Crohn's disease, ulcerative colitis etc.; meningitis; inflammatory ocular disease; inflammatory pulmonary diseases such as pneumonia, pulmonary silicosis, pulmonary sarcoidosis, pulmonary tuberculosis etc.), allergic diseases (e.g., allergic rhinitis, conjunctivitis, gastrointestinal allergy, pollinosis, anaphylaxis etc.), chronic obstructive pulmonary disease, interstitial pneumonia, pneumocytis carinni pneumonia, collagen diseases (e.g., systemic lupus erythematodes, scleroderma, polyarteritis etc.), hepatic diseases (e.g., hepatitis including chronic hepatitis, hepatic cirrhosis etc.), portal hypertension, digestive system disorders (e.g., gastritis, gastric ulcer, gastric cancer, gastric disorder after operation, dyspepsia, esophageal ulcer, pancreatitis, colon polyp, cholelithiasis, hemorrhoidal disease, varices ruptures of esophagus and stomach etc.), blood and/or myelopoietic diseases (e.g., erythrocytosis, vascular purpura, autoimmune hemolytic anemia, disseminated intravascular coagulation syndrome, multiple myelopathy etc.), bone diseases (e.g., fracture, refracture, osteoporosis, osteomalacia, bone Paget's disease, sclerosing myelitis, rheumatoid arthritis, joint tissue dysfunction and the like caused by osteoarthritis of the knee and diseases similar to these), solid tumor, tumors (e.g., malignant melanoma, malignant lymphoma, cancer of digestive organs (e.g., stomach, intestine etc.) etc.), cancer and cachexia following cancer, metastasis cancer, endocrinopathy (e.g., Addison's disease, Cushing's syndrome, pheochromocytoma, hyperaldosteronism, primary aldosteronism etc.), Creutzfeldt-Jakob disease, urinary organ and/or male genital diseases (e.g., cystitis, prostatic hypertrophy, prostatic cancer, sex infectious disease etc.), female disorders (e.g., climacteric disorder, gestosis, endometriosis, hysteromyoma, ovarian disease, breast disease, sex infectious disease etc.), disease relating to environment and occupational factors (e.g., radiation hazard, hazard by ultraviolet, infrared or laser beam, altitude sickness etc.), respiratory diseases (e.g., cold syndrome, pneumonia, asthma, pulmonary hypertension, pulmonary thrombosis and pulmonary embolism etc.), infectious diseases (e.g., viral infectious diseases with cytomegalovirus, influenza virus, herpes virus etc., rickettsiosis, bacterial infectious disease etc.), toxemias (e.g., sepsis, septic shock, endotoxin shock, Gram-negative sepsis, toxic shock syndrome etc.), otorhinolaryngological diseases (e.g., Meniere's syndrome, tinnitus, dysgeusia, vertigo, disequilibrium, dysphagia etc.), skin diseases (e.g., keloid, hemangioma, psoriasis etc.), intradialytic hypotension, myasthenia gravis, systemic diseases such as chronic fatigue syndrome and the like, and the like.

The solid preparation of the present invention comprising a combination of a compound represented by the formula (I) or a salt thereof and a calcium antagonist is useful as a prophylactic or therapeutic drug for the above-mentioned diseases (preferably, a prophylactic or therapeutic drug for hypertension, cardiac failure, diabetic nephropathy or arteriosclerosis, more preferably, a prophylactic or therapeutic drug for hypertension). Moreover, the solid preparation of the present invention can reduce the doses of a compound represented by the formula (I) or a salt thereof and a calcium antagonist as compared to single use thereof.

While the dose of a compound represented by the formula (I) or a salt thereof varies depending on the subject of administration, administration route, target disease, symptom and the like, the daily dose for a human adult (body weight 60 kg) based on a free form is about 0.05 to 500 mg, preferably 0.1 to 100 mg, more preferably 1 to 100 mg, still more preferably 2 to 40 mg. For example, the daily dose of compound A for human adult (body weight 60 kg) is about 1 to 80 mg, preferably 2 to 32 mg, and the daily dose of compound B for human adult (body weight 60 kg) is about 1 to 50 mg, preferably 10 to 40 mg.

While the dose of a calcium antagonist varies depending on the subject of administration, administration route, target disease, symptom and the like, the daily dose of, for example, amlodipine or a salt thereof for a human adult (body weight 60 kg) based on a free form is about 1 to 50 mg, preferably 2.5 to 10 mg.

The administration frequency of the solid preparation of the present invention to the aforementioned mammals is preferably 1 to 3 times per day, more preferably once per day.

Particularly preferable specific examples of the solid preparation of the present invention include

“a single-layer tablet comprising 8 mg of compound A and 6.93 mg of amlodipine besylate (5 mg as amlodipine) per tablet”;
“a single-layer tablet comprising 8 mg of compound A and 3.47 mg of amlodipine besylate (2.5 mg as amlodipine) per tablet”;
“a single-layer tablet comprising 4 mg of compound A and 6.93 mg of amlodipine besylate (5 mg as amlodipine) per tablet”;
“a single-layer tablet comprising 4 mg of compound A and 3.47 mg of amlodipine besylate (2.5 mg as amlodipine) per tablet”;
“a single-layer tablet comprising 40 mg of compound B and 6.93 mg of amlodipine besylate (5 mg as amlodipine) per tablet”;
“a single-layer tablet comprising 40 mg of compound B and 3.47 mg of amlodipine besylate (2.5 mg as amlodipine) per tablet”;
“a single-layer tablet comprising 20 mg of compound B and 6.93 mg of amlodipine besylate (5 mg as amlodipine) per tablet”;
“a single-layer tablet comprising 20 mg of compound B and 3.47 mg of amlodipine besylate (2.5 mg as amlodipine) per tablet”;
“a single-layer tablet comprising 10 mg of compound B and 6.93 mg of amlodipine besylate (5 mg as amlodipine) per tablet”; and
“a single-layer tablet comprising 10 mg of compound B and 3.47 mg of amlodipine besylate (2.5 mg as amlodipine) per tablet”.

The solid preparation of the present invention can be used in combination with one or more different kinds of medicaments (hereinafter sometimes to be abbreviated as “concomitant drug”). Examples of the “concomitant drug” include therapeutic agents for diabetes, therapeutic agents for diabetic complications, therapeutic agents for hyperlipidemia, antihypertensive agents, antiobestic agents, diuretics, antithrombotic agents and the like.

Here, as the therapeutic agent for diabetes, for example, insulin preparations (e.g., animal insulin preparations extracted from the pancreas of bovine or swine; human insulin preparations genetically synthesized using Escherichia coli or yeast; zinc insulin; protamine zinc insulin; fragment or derivative of insulin (e.g., INS-1), oral insulin preparation), insulin sensitizers (e.g., pioglitazone or a salt thereof (preferably, hydrochloride), rosiglitazone or a salt thereof (preferably, maleate), Metaglidasen, AMG-131, Balaglitazone, MBX-2044, Rivoglitazone, Aleglitazar, Chiglitazar, Lobeglitazone, PLX-204, PN-2034, GFT-505, THR-0921, compounds described in WO2007/013694, WO2007/018314, WO2008/093639 or WO2008/099794), α-glucosidase inhibitors (e.g., voglibose, acarbose, miglitol, emiglitate), biguanides (e.g., metformin, buformin or a salt thereof (e.g., hydrochloride, fumarate, succinate)), insulin secretagogues (e.g., sulfonylurea (e.g., tolbutamide, glibenclamide, gliclazide, chlorpropamide, tolazamide, acetohexamide, glyclopyramide, glimepiride, glipizide, glybuzole), repaglinide, nateglinide, mitiglinide or calcium salt hydrate thereof), dipeptidyl peptidase IV inhibitors (e.g., Alogliptin or a salt thereof (preferably, benzoate), Vildagliptin, Sitagliptin, Saxagliptin, BI1356, GRC8200, MP-513, PF-00734200, PHX1149, SK-0403, ALS2-0426, TA-6666, TS-021, KRP-104, 2-[[6-[(3R)-3-amino-1-piperidinyl]-3,4-dihydro-3-methyl-2,4-dioxo-1(2H)-pyrimidinyl]methyl]-4-fluorobenzonitrile or a salt thereof), β3 agonists (e.g., N-5984), GPR40 agonists (e.g., compounds described in WO2004/041266, WO2004/106276, WO2005/063729, WO2005/063725, WO2005/087710, WO2005/095338, WO2007/013689 or WO2008/001931), GLP-1 receptor agonists (e.g., GLP-1, GLP-1MR agent, Liraglutide, Exenatide, AVE-0010, BIM-51077, Aib(8,35)hGLP-1(7,37)NH2, CJC-1131, Albiglutide), amylin agonists (e.g., pramlintide), phosphotyrosine phosphatase inhibitors (e.g., sodium vanadate), gluconeogenesis inhibitors (e.g., glycogen phosphorylase inhibitors, glucose-6-phosphatase inhibitors, glucagon antagonists, FBPase inhibitors), SGLT2 (sodium-glucose cotransporter 2) inhibitors (e.g., Depagliflozin, AVE2268, TS-033, YM543, TA-7284, Remogliflozin, ASP1941), SGLT1 inhibitors, 11β-hydroxysteroid dehydrogenase inhibitors (e.g., BVT-3498, INCB-13739), adiponectin or agonist thereof, IKK inhibitors (e.g., AS-2868), leptin resistance improving drugs, somatostatin receptor agonists, glucokinase activators (e.g., Piragliatin, AZD1656, AZD6370, TTP-355, compounds described in WO2006/112549, WO2007/028135, WO2008/047821, WO2008/050821, WO2008/136428 or WO2008/156757), GIP (Glucose-dependent insulinotropic peptide), GPR119 agonists (e.g., PSN821), FGF21, FGF analogue and the like can be mentioned.

As the therapeutic agent for diabetic complications, aldose reductase inhibitors (e.g., tolrestat, epalrestat, zopolrestat, fidarestat, CT-112, ranirestat (AS-3201), lidorestat), neurotrophic factor and increasing agents thereof (e.g., NGF, NT-3, BDNF, neurotrophic production/secretion promoting agent described in WO01/14372 (e.g., 4-(4-chlorophenyl)-2-(2-methyl-1-imidazolyl)-5-[3-(2-methylphenoxy)propyl]oxazole), compounds described in WO2004/039365), PKC inhibitors (e.g., ruboxistaurin mesylate), AGE inhibitors (e.g., ALT946, N-phenacylthiazolium bromide (ALT766), EXO-226, Pyridorin, pyridoxamine), GABA receptor agonists (e.g., gabapentin, pregabalin), serotonin and norepinephrine reuptake inhibitors (e.g., duloxetine), sodium channel inhibitors (e.g., lacosamide), active oxygen scavengers (e.g., thioctic acid), cerebral vasodilators (e.g., tiapuride, mexiletine), somatostatin receptor agonists (e.g., BIM23190), apoptosis signal regulating kinase-1 (ASK-1) inhibitors and the like can be mentioned.

As the therapeutic agent for hyperlipidemia, HMG-CoA reductase inhibitors (e.g., pravastatin, simvastatin, lovastatin, atorvastatin, fluvastatin, rosuvastatin, pitavastatin or a salt thereof (e.g., sodium salt, calcium salt)), squalene synthase inhibitors (e.g., compounds described in WO97/10224, for example, N-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidin-4-acetic acid), fibrate compounds (e.g., bezafibrate, clofibrate, simfibrate, clinofibrate), anion exchange resin (e.g., colestyramine), probucol, nicotinic acid drugs (e.g., nicomol, niceritrol, niaspan), ethyl icosapentate, phytosterol (e.g., soysterol, gamma oryzanol (γ-oryzanol)), cholesterol absorption inhibitors (e.g., zechia), CETP inhibitors (e.g., dalcetrapib, anacetrapib), ω-3 fatty acid preparations (e.g., ω-3-acid ethyl esters 90) and the like can be mentioned.

As the antihypertensive agent, angiotensin converting enzyme inhibitors (e.g., captopril, enalapril, delapril and the like), angiotensin II antagonists (e.g., candesartan cilexetil, candesartan, losartan, losartan potassium, eprosartan, valsartan, telmisartan, irbesartan, tasosartan, olmesart, olmesartan medoxomil, azilsartan and the like), a calcium antagonists (e.g., manidipine, nifedipine, amlodipine, efonidipine, nicardipine, amlodipine, cilnidipine and the like), β blockers (e.g., metoprolol, atenolol, propranolol, carvedilol, pindolol and the like), clonidine and the like can be mentioned.

As the antiobesity agent, monoamine uptake inhibitors (e.g., phentermine, sibutramine, mazindol, fluoxetine, tesofensine), serotonin 2C receptor agonists (e.g., lorcaserin), serotonin 6 receptor antagonists, histamine H3 receptor, GABA-regulating drugs (e.g., topiramate), neuropeptide Y antagonists (e.g., velneperit), cannabinoid receptor antagonists (e.g., rimonabant, taranabant), ghrelinant agonists, ghrelin receptor antagonists, ghrelin acylation enzyme inhibitors, opioid receptor antagonists (e.g., GSK-1521498), orexin receptor antagonists, melanocortin 4 receptor agonists, 11β-hydroxysteroid dehydrogenase inhibitors (e.g., AZD-4017), pancreatic lipase inhibitors (e.g., orlistat, cetilistat), β3 agonists (e.g., N-5984), diacylglycerol acyltransferase 1 (DGAT1) inhibitors, acetyl CoA carboxylase (ACC) inhibitors, steaoryl-CoA desaturase inhibitors, microsomal triglyceride transfer protein inhibitors (e.g., R-256918), Na-glucose cotransporter inhibitors (e.g., JNJ-28431754, remogliflozin), NFκB inhibitors (e.g., HE-3286), PPAR agonists (e.g., GFT-505, DRF-11605), phosphotyrosine phosphatase inhibitors (e.g., sodium vanadate, Trodusquemin), GPR119 agonists (e.g., PSN-821), glucokinase activators (e.g., AZD-1656), leptin, leptin derivatives (e.g., metreleptin), CNTF (ciliary neurotrophic factor), BDNF (brain-derived neurotrophic factor), cholecystokinin agonists, glucagon-like peptide-1 (GLP-1) preparations (e.g., animal GLP-1 preparations extracted from the pancreas of bovine or swine; human GLP-1 preparations genetically synthesized using Escherichia coli or yeast; fragment or derivative of GLP-1 (e.g., exenatide, liraglutide)), amylin preparations (e.g., pramlintide, AC-2307), neuropeptide Y agonists (e.g., PYY3-36, derivative of PYY3-36, obinepitide, TM-30339, TM-30335), oxyntomodulin preparations; FGF21 preparations (e.g., animal FGF21 preparations extracted from the pancreas of bovine or swine; human FGF21 preparations genetically synthesized using Escherichia coli or yeast; fragment or derivative of FGF21)), feeding deterrents (e.g., P-57) and the like can be mentioned.

As the diuretic, for example, xanthine derivatives (e.g., theobromine sodium salicylate, theobromine calcium salicylate and the like), thiazide preparations (e.g., ethiazide, cyclopenthiazide, trichloromethyazide, hydrochlorothiazide, hydroflumethiazide, benzylhydrochlorothiazide, penfluthiazide, poly5thiazide, methyclothiazide and the like), antialdosterone preparations (e.g., spironolactone, triamterene and the like), carbonic anhydrase inhibitors (e.g., acetazolamide and the like), chlorobenzenesulfonamide agents (e.g., chlortalidone, mefruside, indapamide and the like), azosemide, isosorbide, ethacrynic acid, piretanide, bumetanide, furosemide and the like can be mentioned.

As the antithrombotic agents, for example, heparin (e.g., heparin sodium, heparin calcium, enoxaparin sodium, dalteparin sodium), warfarin (e.g., warfarin potassium), anti-thrombin drugs (e.g., aragatroban, dabigatran), FXa inhibitors (e.g., rivaroxaban, apixaban, edoxaban, YM150, compounds described in WO02/06234, WO2004/048363, WO2005/030740, WO2005/058823 or WO2005/113504), thrombolytic agents (e.g., urokinase, tisokinase, alteplase, nateplase, monteplase, pamiteplase), platelet aggregation inhibitors (e.g., ticlopidine hydrochloride, clopidogrel, prasugrel, E5555, SHC530348, cilostazol, ethyl icosapentate, beraprost sodium, sarpogrelate hydrochloride) and the like can be mentioned.

When the solid preparation of the present invention and a concomitant drug are used in combination, the administration period thereof is not limited, and they may be administered simultaneously or in a staggered manner to the administration subject.

Moreover, the solid preparation of the present invention and a concomitant drug may be administered as separate preparations, or as a single preparation containing the solid preparation of the present invention and a concomitant drug.

The dose of the concomitant drug can be appropriately determined based on the dose employed in clinical situations of each drug. The mixing ratio of the solid preparation of the present invention and a concomitant drug can be appropriately determined depending on the administration subject, administration route, target disease, symptom, combination and the like. When the subject of administration is human, for example, a concomitant drug can be used in 0.01 to 100 parts by weight relative to 1 part by weight of the solid preparation of the present invention.

Using a concomitant drug in this way, superior effects of

1) an effect of enhancing the action of the solid preparation of the present invention or a concomitant drug (synergistic effect of medicament actions);
2) an effect of reducing the dose of the solid preparation of the present invention or a concomitant drug (medicament dose-reducing effect as compared to single administration);
3) an effect of reducing the secondary action of the solid preparation of the present invention or a concomitant drug; and the like can be obtained.

The present invention also provides a method of improving the dissolution property of and a method of stabilizing a compound represented by the formula (I) or a salt thereof, and/or a calcium antagonist, contained in a solid preparation, which comprises adding sugar alcohol. According to the present invention, the dissolution property of a compound represented by the formula (I) or a salt thereof in the solid preparation are significantly improved.

### EXAMPLES

The present invention is explained in more detail in the following by referring to Examples and Experimental Examples, which are not to be construed as limitative.

In the following Examples and Experimental Examples, as the components (additives) other than the active ingredient, the Japanese Pharmacopoeia 15th Revision, the Japanese Pharmacopoeia Japanese Pharmaceutical Codex or Japanese Pharmaceutical Excipients 2003 compatible products were used.

In the following Examples, as a compound represented by the above-mentioned formula (I) or a salt thereof, compound A or compound B was used.

### Example 1

TABLE 1
Composition per preparation (130 mg)

| compound A | 8 | mg |
|---|---|---|
| amlodipine besylate | 6.93 | mg |
| | (5 mg as amlodipine) | |
| D-mannitol | 82.754 | mg |
| microcrystalline cellulose | 20 | mg |
| hydroxypropylcellulose | 4 | mg |
| macrogol 6000 | 1.8 | mg |
| croscarmellose sodium | 5.6 | mg |
| magnesium stearate | 0.9 | mg |
| red ferric oxide | 0.016 | mg |
| total | 130 | mg |

(1) Hydroxypropylcellulose (720.0 g) and macrogol 6000 (324.0 g) were dissolved in purified water (9000 g) to give binding solution I. Red ferric oxide (2.880 g) was dispersed in purified water (2880 g) to give dispersion I. Binding solution I was mixed with dispersion I and purified water (720.0 g) to give binding solution II. Amlodipine besylate (1253 g), compound A (1449 g), D-mannitol (14880 g) and microcrystalline cellulose (3600 g) were uniformly mixed in a fluid bed granulator (FD-S2, POWREX Co., Ltd.), and the mixture was granulated while spraying binding solution II, and then dried to give a granule. A part of the obtained granule was milled with a 1.5 mmφ punching screen in a screening mill (P-3, Showa Kagakukikai Co., Ltd.) to give a milled granule.
(2) To the obtained milled granule (40760 g (2 batches)) were added croscarmellose sodium (1848 g) and magnesium stearate (297.0 g), and they were mixed in a tumbler mixer (TM20-0-0, Suchiro Kakoki Co., Ltd.) to give a mixed granule.
(3) The mixed granule obtained above was tableted by a rotary tableting machine (AQUARIUS-36K, Kikusui Seisakusho) with a diameter of 7.0 mm punch (tableting pressure 10 kN, weight per tablet: 130 mg) to give plain tablets of the composition shown in Table 1.

### Example 2

TABLE 2
Composition per preparation (130 mg)

| compound A | 8 | mg |
|---|---|---|
| amlodipine besylate | 6.93 | mg |
| | (5 mg as amlodipine) | |
| D-mannitol | 82.705 | mg |
| microcrystalline cellulose | 20 | mg |
| hydroxypropylcellulose | 4 | mg |
| macrogol 6000 | 1.8 | mg |
| croscarmellose sodium | 5.6 | mg |
| magnesium stearate | 0.9 | mg |
| red ferric oxide | 0.065 | mg |
| total | 130 | mg |

(1) Hydroxypropylcellulose (720.0 g) and macrogol 6000 (324.0 g) were dissolved in purified water (9900 g) to give binding solution I. Red ferric oxide (11.70 g) was dispersed in purified water (1800 g) to give dispersion I. Binding solution I was mixed with dispersion I and purified water (540.0 g) to give binding solution II. Amlodipine besylate (1253 g), compound A (1449 g), D-mannitol (14870 g) and microcrystalline cellulose (3600 g) were uniformly mixed in a fluid bed granulator (FD-S2, POWREX Co., Ltd.), and the mixture was granulated while spraying binding solution II, and then dried to give a granule. A part of the obtained granule was milled with a 1.5 mmφ punching screen in a screening mill (P-3, Showa Kagakukikai Co., Ltd.) to give a milled granule.
(2) To the obtained milled granule (40760 g (2 batches)) were added croscarmellose sodium (1848 g) and magnesium stearate (297.0 g), and they were mixed in a tumbler mixer (TM20-0-0, Suchiro Kakoki Co., Ltd.) to give a mixed granule.
(3) The mixed granule obtained above was tableted by a rotary tableting machine (AQUARIUS-36K, Kikusui Seisakusho) with a long diameter of 8.5 mm and short diameter of 5.0 mm punch (tableting pressure 8 kN, weight per tablet: 130 mg) to give plain tablets of the composition shown in Table 2.

### Example 3

TABLE 3
Composition per preparation (130 mg)

| compound A | 8 | mg |
|---|---|---|
| amlodipine besylate | 3.47 | mg |
| | (2.5 mg as amlodipine) | |
| D-mannitol | 86.165 | mg |
| microcrystalline cellulose | 20 | mg |
| hydroxypropylcellulose | 4 | mg |
| macrogol 6000 | 1.8 | mg |
| croscarmellose sodium | 5.6 | mg |
| magnesium stearate | 0.9 | mg |
| yellow ferric oxide | 0.065 | mg |
| total | 130 | mg |

(1) Hydroxypropylcellulose (720.0 g) and macrogol 6000 (324.0 g) were dissolved in purified water (9900 g) to give binding solution I. Yellow ferric oxide (11.70 g) was dispersed in purified water (1800 g) to give dispersion I. Binding solution I was mixed with dispersion I and purified water (540.0 g) to give binding solution II. Amlodipine besylate (627.7 g), compound A (1449 g), D-mannitol (15550 g) and microcrystalline cellulose (3600 g) were uniformly mixed in a fluid bed granulator (FD-S2, POWREX Co., Ltd.), and the mixture was granulated while spraying binding solution II, and then dried to give a granule. A part of the obtained granule was milled with a 1.5 mmφ punching screen in a screening mill (P-3, Showa Kagakukikai Co., Ltd.) to give a milled granule.
(2) To the obtained milled granule (40760 g (2 batches)) were added croscarmellose sodium (1848 g) and magnesium stearate (297.0 g), and they were mixed in a tumbler mixer (TM20-0-0, Suchiro Kakoki Co., Ltd.) to give a mixed granule.
(3) The mixed granule obtained above was tableted by a rotary tableting machine (AQUARIUS-36K, Kikusui Seisakusho) with a long diameter of 8.5 mm and short diameter of 5.0 mm punch (tableting pressure 8 kN, weight per tablet: 130 mg) to give plain tablets of the composition shown in Table 3.

### Example 4

TABLE 4
Composition per preparation (130 mg)

| compound A | 4 | mg |
|---|---|---|
| amlodipine besylate | 6.93 | mg |
| | (5 mg as amlodipine) | |
| D-mannitol | 86.705 | mg |
| microcrystalline cellulose | 20 | mg |
| hydroxypropylcellulose | 4 | mg |
| macrogol 6000 | 1.8 | mg |
| croscarmellose sodium | 5.6 | mg |
| magnesium stearate | 0.9 | mg |
| red ferric oxide | 0.065 | mg |
| total | 130 | mg |

(1) Hydroxypropylcellulose (720.0 g) and macrogol 6000 (324.0 g) were dissolved in purified water (9900 g) to give binding solution I. Red ferric oxide (11.70 g) was dispersed in purified water (1800 g) to give dispersion I. Binding solution I was mixed with dispersion I and purified water (540.0 g) to give binding solution II. Amlodipine besylate (1253 g), compound A (724.3 g), D-mannitol (15600 g) and microcrystalline cellulose (3600 g) were uniformly mixed in a fluid bed granulator (FD-S2, POWREX Co., Ltd.), and the mixture was granulated while spraying binding solution II, and then dried to give a granule. A part of the obtained granule was milled with a 1.5 mmφ punching screen in a screening mill (P-3, Showa Kagakukikai Co., Ltd.) to give a milled granule.
(2) To the obtained milled granule (40760 g (2 batches)) were added croscarmellose sodium (1848 g) and magnesium stearate (297.0 g), and they were mixed in a tumbler mixer (TM20-0-0, Suchiro Kakoki Co., Ltd.) to give a mixed granule.
(3) The mixed granule obtained above was tableted by a rotary tableting machine (AQUARIUS-36K, Kikusui Seisakusho) with a diameter of 7 mm punch (tableting pressure 10 kN, weight per tablet: 130 mg) to give plain tablets of the composition shown in Table 4.

### Example 5

TABLE 5
Composition per preparation (130 mg)

| compound A | 4 | mg |
|---|---|---|
| amlodipine besylate | 3.47 | mg |
| | (2.5 mg as amlodipine) | |
| D-mannitol | 90.165 | mg |
| microcrystalline cellulose | 20 | mg |
| hydroxypropylcellulose | 4 | mg |
| macrogol 6000 | 1.8 | mg |
| croscarmellose sodium | 5.6 | mg |
| magnesium stearate | 0.9 | mg |
| yellow ferric oxide | 0.065 | mg |
| total | 130 | mg |

(1) Hydroxypropylcellulose (720.0 g) and macrogol 6000 (324.0 g) were dissolved in purified water (9900 g) to give binding solution I. Yellow ferric oxide (11.70 g) was dispersed in purified water (1800 g) to give dispersion I. Binding solution I was mixed with dispersion I and purified water (540.0 g) to give binding solution II. Amlodipine besylate (627.7 g), compound A (724.3 g), D-mannitol (16220 g) and microcrystalline cellulose (3600 g) were uniformly mixed in a fluid bed granulator (FD-S2, POWREX Co., Ltd.), and the mixture was granulated while spraying binding solution II, and then dried to give a granule. A part of the obtained granule was milled with a 1.5 mmφ punching screen in a screening mill (P-3, Showa Kagakukikai Co., Ltd.) to give a milled granule.
(2) To the obtained milled granule (40760 g (2 batches)) were added croscarmellose sodium (1848 g) and magnesium stearate (297.0 g), and they were mixed in a tumbler mixer (TM20-0-0, Suchiro Kakoki Co., Ltd.) to give a mixed granule.
(3) The mixed granule obtained above was tableted by a rotary tableting machine (AQUARIUS-36K, Kikusui Seisakusho) with a diameter of 7 mm punch (tableting pressure 10 kN, weight per tablet: 130 mg) to give plain tablets of the composition shown in Table 5.

### Example 6

TABLE 6
Composition per preparation (130 mg)

| compound A | 8 | mg |
|---|---|---|
| amlodipine besylate | 6.93 | mg |
| | (5 mg as amlodipine) | |
| D-mannitol | 82.666 | mg |
| microcrystalline cellulose | 20 | mg |
| hydroxypropylcellulose | 4 | mg |
| macrogol 6000 | 1.8 | mg |
| croscarmellose sodium | 5.6 | mg |
| magnesium stearate | 0.9 | mg |
| red ferric oxide | 0.104 | mg |
| total | 130 | mg |

(1) Hydroxypropylcellulose (720.0 g) and macrogol 6000 (324.0 g) were dissolved in purified water (9900 g) to give binding solution I. Red ferric oxide (18.72 g) was dispersed in purified water (1800 g) to give dispersion I. Binding solution I was mixed with dispersion I and purified water (540.0 g) to give binding solution II. Amlodipine besylate (1248 g), compound A (1446 g), D-mannitol (14870 g) and microcrystalline cellulose (3600 g) were uniformly mixed in a fluid bed granulator (FD-S2, POWREX Co., Ltd.), and the mixture was granulated while spraying binding solution II, and then dried to give a granule. A part of the obtained granule was milled with a 1.5 mmφ punching screen in a screening mill (P-3, Showa Kagakukikai Co., Ltd.) to give a milled granule.
(2) To the obtained milled granule (20380 g) were added croscarmellose sodium (924.0 g) and magnesium stearate (148.5 g), and they were mixed in a tumbler mixer (TM20-0-0, Suchiro Kakoki Co., Ltd.) to give a mixed granule.
(3) The mixed granule obtained above was tableted by a rotary tableting machine (AQUARIUS-36K, Kikusui Seisakusho) with a long diameter of 8.5 mm and short diameter of 5.0 mm punch (tableting pressure 8 kN, weight per tablet: 130 mg) to give plain tablets of the composition shown in Table 6.

### Example 7

TABLE 7
Composition per preparation (130 mg)

| compound A | 8 | mg |
|---|---|---|
| amlodipine besylate | 3.47 | mg |
| | (2.5 mg as amlodipine) | |
| D-mannitol | 86.126 | mg |
| microcrystalline cellulose | 20 | mg |
| hydroxypropylcellulose | 4 | mg |
| macrogol 6000 | 1.8 | mg |
| croscarmellose sodium | 5.6 | mg |
| magnesium stearate | 0.9 | mg |
| yellow ferric oxide | 0.104 | mg |
| total | 130 | mg |

(1) Hydroxypropylcellulose (720.0 g) and macrogol 6000 (324.0 g) were dissolved in purified water (9900 g) to give binding solution I. Yellow ferric oxide (18.72 g) was dispersed in purified water (1800 g) to give dispersion I. Binding solution I was mixed with dispersion I and purified water (540.0 g) to give binding solution II. Amlodipine besylate (625.2 g), compound A (1446 g), D-mannitol (15500 g) and microcrystalline cellulose (3600 g) were uniformly mixed in a fluid bed granulator (FD-S2, POWREX Co., Ltd.), and the mixture was granulated while spraying binding solution II, and then dried to give a granule. A part of the obtained granule was milled with a 1.5 mmφ punching screen in a screening mill (P-3, Showa Kagakukikai Co., Ltd.) to give a milled granule.
(2) To the obtained milled granule (20380 g) were added croscarmellose sodium (924.0 g) and magnesium stearate (148.5 g), and they were mixed in a tumbler mixer (TM20-0-0, Suchiro Kakoki Co., Ltd.) to give a mixed granule.
(3) The mixed granule obtained above was tableted by a rotary tableting machine (AQUARIUS-36K, Kikusui Seisakusho) with a long diameter of 8.5 mm and short diameter of 5.0 mm punch (tableting pressure 8 kN, weight per tablet: 130 mg) to give plain tablets of the composition shown in Table 7.

### Example 8

TABLE 8
Composition per preparation (130 mg)

| compound A | 8 | mg |
|---|---|---|
| amlodipine besylate | 6.93 | mg |
| | (5 mg as amlodipine) | |
| D-mannitol | 83.166 | mg |
| microcrystalline cellulose | 20 | mg |
| hydroxypropylcellulose | 4 | mg |
| macrogol 6000 | 1.3 | mg |
| croscarmellose sodium | 5.6 | mg |
| magnesium stearate | 0.9 | mg |
| red ferric oxide | 0.104 | mg |
| total | 130 | mg |

(1) Hydroxypropylcellulose (144.0 g) was dissolved in purified water (1980 g) to give binding solution I. Yellow ferric oxide (3.744 g) was dispersed in purified water (360.0 g) to give dispersion I. Binding solution I was mixed with dispersion I and purified water (108.0 g) to give binding solution II. Macrogol 6000 (4.680 g) was dissolved in binding solution II (259.6 g) to give binding solution III. Amlodipine besylate (24.95 g), compound A (28.80 g), D-mannitol (299.4 g) and microcrystalline cellulose (72.00 g) were uniformly mixed in a fluid bed granulator (Lab-1, POWREX Co., Ltd.), and the mixture was granulated while spraying binding solution III, and then dried to give a granule. A part of the obtained granule was sieved with a sieve (16 mesh) to give a milled granule.
(2) To the obtained milled granule (370.5 g) were added croscarmellose sodium (16.80 g) and magnesium stearate (2.700 g), and they were mixed in a polyethylene bag (4.9 L) to give a mixed granule.
(3) The mixed granule obtained above was tableted by a rotary tableting machine (Correct 19K, Kikusui Seisakusho) with a long diameter of 8.5 mm and short diameter of 5.0 mm punch (tableting pressure 7.5 kN, weight per tablet: 130 mg) to give plain tablets of the composition shown in Table 8.

### Example 9

TABLE 9
Composition per preparation (130 mg)

| compound A | 8 | mg |
|---|---|---|
| amlodipine besylate | 6.93 | mg |
| | (5 mg as amlodipine) | |
| D-mannitol | 80.566 | mg |
| microcrystalline cellulose | 20 | mg |
| hydroxypropylcellulose | 4 | mg |
| macrogol 6000 | 3.9 | mg |
| croscarmellose sodium | 5.6 | mg |
| magnesium stearate | 0.9 | mg |
| red ferric oxide | 0.104 | mg |
| total | 130 | mg |

(1) Hydroxypropylcellulose (144.0 g) was dissolved in purified water (1980 g) to give binding solution I. Red ferric oxide (3.744 g) was dispersed in purified water (360.0 g) to give dispersion I. Binding solution I was mixed with dispersion I and purified water (108.0 g) to give binding solution II. Macrogol 6000 (14.04 g) was dissolved in binding solution II (259.6 g) to give binding solution III. Amlodipine besylate (24.95 g), compound A (28.80 g), D-mannitol (290.0 g) and microcrystalline cellulose (72.00 g) were uniformly mixed in a fluid bed granulator (Lab-1, POWREX Co., Ltd.), and the mixture was granulated while spraying binding solution III, and then dried to give a granule. A part of the obtained granule was sieved with a sieve (16 mesh) to give a milled granule.
(2) To the obtained milled granule (370.5 g) were added croscarmellose sodium (16.80 g) and magnesium stearate (2.700 g), and they were mixed in a polyethylene bag (4.9 L) to give a mixed granule.
(3) The mixed granule obtained above was tableted by a rotary tableting machine (Correct 19K, Kikusui Seisakusho) with a long diameter of 8.5 mm and short diameter of 5.0 mm punch (tableting pressure 7.5 kN, weight per tablet: 130 mg) to give plain tablets of the composition shown in Table 9.

### Example 10

TABLE 10
Composition per preparation (130 mg)

| compound A | 8 | mg |
|---|---|---|
| amlodipine besylate | 6.93 | mg |
| | (5 mg as amlodipine) | |
| D-mannitol | 77.966 | mg |
| microcrystalline cellulose | 20 | mg |
| hydroxypropylcellulose | 4 | mg |
| macrogol 6000 | 6.5 | mg |
| croscarmellose sodium | 5.6 | mg |
| magnesium stearate | 0.9 | mg |
| red ferric oxide | 0.104 | mg |
| total | 130 | mg |

(1) Hydroxypropylcellulose (144.0 g) was dissolved in purified water (1980 g) to give binding solution I. Red ferric oxide (3.744 g) was dispersed in purified water (360.0 g) to give dispersion I. Binding solution I was mixed with dispersion I and purified water (108.0 g) to give binding solution II. Macrogol 6000 (23.40 g) was dissolved in binding solution II (259.6 g) to give binding solution III. Amlodipine besylate (24.95 g), compound A (28.80 g), D-mannitol (280.7 g) and microcrystalline cellulose (72.00 g) were uniformly mixed in a fluid bed granulator (Lab-1, POWREX Co., Ltd.), and the mixture was granulated while spraying binding solution III, and then dried to give a granule. A part of the obtained granule was sieved with a sieve (16 mesh) to give a milled granule.
(2) To the obtained milled granule (370.5 g) were added croscarmellose sodium (16.80 g) and magnesium stearate (2.700 g), and they were mixed in a polyethylene bag (4.9 L) to give a mixed granule.
(3) The mixed granule obtained above was tableted by a rotary tableting machine (Correct 19K, Kikusui Seisakusho) with a long diameter of 8.5 mm and short diameter of 5.0 mm punch (tableting pressure 7.5 kN, weight per tablet: 130 mg) to give plain tablets of the composition shown in Table 10.

### Example 11

TABLE 11
Composition per preparation (130 mg)

| compound A | 8 | mg |
|---|---|---|
| amlodipine besylate | 6.93 | mg |
| | (5 mg as amlodipine) | |
| D-mannitol | 74.066 | mg |
| microcrystalline cellulose | 20 | mg |
| hydroxypropylcellulose | 4 | mg |
| macrogol 6000 | 10.4 | mg |
| croscarmellose sodium | 5.6 | mg |
| magnesium stearate | 0.9 | mg |
| red ferric oxide | 0.104 | mg |
| total | 130 | mg |

(1) Hydroxypropylcellulose (144.0 g) was dissolved in purified water (1980 g) to give binding solution I. Red ferric oxide (3.744 g) was dispersed in purified water (360.0 g) to give dispersion I. Binding solution I was mixed with dispersion I and purified water (108.0 g) to give binding solution II. Macrogol 6000 (37.44 g) was dissolved in binding solution II (259.6 g) to give binding solution III. Amlodipine besylate (24.95 g), compound A (28.80 g), D-mannitol (266.6 g) and microcrystalline cellulose (72.00 g) were uniformly mixed in a fluid bed granulator (Lab-1, POWREX Co., Ltd.), and the mixture was granulated while spraying binding solution III, and then dried to give a granule. A part of the obtained granule was sieved with a sieve (16 mesh) to give a milled granule.
(2) To the obtained milled granule (370.5 g) were added croscarmellose sodium (16.80 g) and magnesium stearate (2.700 g), and they were mixed in a polyethylene bag (4.9 L) to give a mixed granule.
(3) The mixed granule obtained above was tableted by a rotary tableting machine (Correct 19K, Kikusui Seisakusho) with a long diameter of 8.5 mm and short diameter of 5.0 mm punch (tableting pressure 7.5 kN, weight per tablet: 130 mg) to give plain tablets of the composition shown in Table 11.

### Example 12

TABLE 12
Composition per preparation (135 mg)

| Compound B | 40 | mg |
|---|---|---|
| amlodipine besylate | 6.93 | mg |
| | (5 mg as amlodipine) | |
| D-mannitol | 40.1 | mg |
| microcrystalline cellulose | 9.72 | mg |
| hydroxypropylcellulose | 5.2 | mg |
| macrogol 6000 | 4 | mg |
| crospovidone | 9.75 | mg |
| microcrystalline cellulose | 13 | mg |
| magnesium stearate | 1.3 | mg |
| premix I | 5 | mg |
| total | 135 | mg |

(1) Hydroxypropylcellulose (208.0 g) and macrogol 6000 (160.0 g) were dissolved in purified water (2392.0 g) to give a binding solution. Amlodipine besylate (277.2 g), compound B (1605.0 g), D-mannitol (1599.0 g) and microcrystalline cellulose (388.8 g) were uniformly mixed in a fluid bed granulator (FD-5S, POWREX Co., Ltd.), and the mixture was granulated while spraying the binding solution, and then dried to give a granule. A part of the obtained granule was milled with a 1.5 mmφ punching screen in a screening mill (P-3, Showa Kagakukikai Co., Ltd.) to give a milled granule.
(2) To the obtained milled granule (3602.0 g) were added crospovidone (331.5 g), microcrystalline cellulose (442.0 g) and magnesium stearate (44.201 g), and they were mixed in a tumbler mixer (TM-15, Suchiro Kakoki co., Ltd.) to give a mixed granule.
(3) The mixed granule obtained above was tableted by a rotary tableting machine (Correct 12HUK, Kikusui Seisakusho) with a diameter of 7.0 mm punch (tableting pressure 4 kN, weight per tablet: 130 mg) to give core tablets.
(4) Premix I (240.0 g) was dissolved in purified water (2160.0 g) to give a film coating solution. A film coating was formed by uniformly spraying the film coating solution on the core tablets (3120.0 g) in a film coating machine (DRC-500, POWREX Co., Ltd.) to give film-coated tablets of the composition shown in Table 12 (weight per tablet: 135 mg). Here, the premix I is a premixed powder. The composition of the premix I is shown in Table 12a.

TABLE 12a
Composition of premix I

| | ratio by weight |
|---|---|
| hypromellose | 9.0 |
| macrogol 6000 | 2.0 |
| titanium dioxide | 1.0 |
| yellow ferric oxide | 0.2 |

### Example 13

TABLE 13
Composition per preparation (135 mg)

| Compound B | 20 | mg |
|---|---|---|
| amlodipine besylate | 6.93 | mg |
| | (5 mg as amlodipine) | |
| D-mannitol | 60.1 | mg |
| microcrystalline cellulose | 9.72 | mg |
| hydroxypropylcellulose | 5.2 | mg |
| macrogol 6000 | 4 | mg |
| crospovidone | 9.75 | mg |
| microcrystalline cellulose | 13 | mg |
| magnesium stearate | 1.3 | mg |
| premix I | 5 | mg |
| total | 135 | mg |

(1) Hydroxypropylcellulose (20.80 g) and macrogol 6000 (16.00 g) were dissolved in purified water (239.2 g) to give a binding solution. Amlodipine besylate (27.72 g), compound B (80.00 g), D-mannitol (240.4 g) and microcrystalline cellulose (38.88 g) were uniformly mixed in a fluid bed granulator (Lab-1, POWREX Co., Ltd.), and the mixture was granulated while spraying the binding solution, and then dried to give a granule. A part of the obtained granule was sieved with a sieve (16 mesh) to give a milled granule.
(2) To the obtained milled granule (370.8 g) were added crospovidone (34.13 g), microcrystalline cellulose (45.50 g) and magnesium stearate (4.550 g), and they were mixed in a polyethylene bag (4.9 L) to give a mixed granule.
(3) The mixed granule obtained above was tableted by a rotary tableting machine (VEL-5, Kikusui Seisakusho) with a diameter of 7.0 mm punch (tableting pressure 4 kN, weight per tablet: 130 mg) to give core tablets.
(4) Premix I (40.00 g) was dissolved in purified water (360.0 g) to give a film coating solution. A film coating was formed by uniformly spraying the film coating solution on the core tablets (200.0 g) in a film coating machine (DRC-200, POWREX Co., Ltd.) to give film-coated tablets of the composition shown in Table 13 (weight per tablet: 135 mg). Here, the premix I is a premixed powder. The composition of the premix I is shown in Table 13a.

TABLE 13a
Composition of premix I

| | ratio by weight |
|---|---|
| hypromellose | 9.0 |
| macrogol 6000 | 2.0 |
| titanium dioxide | 1.0 |
| yellow ferric oxide | 0.2 |

### Example 14

TABLE 14
Composition per preparation (135 mg)

| Compound B | 20 | mg |
|---|---|---|
| amlodipine besylate | 3.47 | mg |
| | (2.5 mg as amlodipine) | |
| D-mannitol | 63.56 | mg |
| microcrystalline cellulose | 9.72 | mg |
| hydroxypropylcellulose | 5.2 | mg |
| macrogol 6000 | 4 | mg |
| crospovidone | 9.75 | mg |
| microcrystalline cellulose | 13 | mg |
| magnesium stearate | 1.3 | mg |
| premix I | 5 | mg |
| total | 135 | mg |

(1) Hydroxypropylcellulose (20.80 g) and macrogol 6000 (16.00 g) were dissolved in purified water (239.2 g) to give a binding solution. Amlodipine besylate (13.88 g), compound B (80.00 g), D-mannitol (254.2 g) and microcrystalline cellulose (38.88 g) were uniformly mixed in a fluid bed granulator (Lab-1, POWREX Co., Ltd.), and the mixture was granulated while spraying the binding solution, and then dried to give a granule. A part of the obtained granule was sieved with a sieve (16 mesh) to give a milled granule.
(2) To the obtained milled granule (370.8 g) were added crospovidone (34.13 g), microcrystalline cellulose (45.50 g) and magnesium stearate (4.550 g), and they were mixed in a polyethylene bag (4.9 L) to give a mixed granule.
(3) The mixed granule obtained above was tableted by a rotary tableting machine (VEL-5, Kikusui Seisakusho) with a diameter of 7.0 mm punch (tableting pressure 4 kN, weight per tablet: 130 mg) to give core tablets.
(4) Premix I (40.00 g) was dissolved in purified water (360.0 g) to give a film coating solution. A film coating was formed by uniformly spraying the film coating solution on the core tablets (200.0 g) in a film coating machine (DRC-200, POWREX Co., Ltd.) to give film-coated tablets of the composition shown in Table 14 (weight per tablet: 135 mg). Here, the premix I is a premixed powder. The composition of the premix I is shown in Table 14a.

TABLE 14a
Composition of premix I

| | ratio by weight |
|---|---|
| hypromellose | 9.0 |
| macrogol 6000 | 2.0 |
| titanium dioxide | 1.0 |
| yellow ferric oxide | 0.2 |

### Example 15

TABLE 15
Composition per preparation (135.154 mg)

| Compound B | 10 | mg |
|---|---|---|
| amlodipine besylate | 3.465 | mg |
| | (5 mg as amlodipine) | |
| D-mannitol | 83.535 | mg |
| microcrystalline cellulose | 13 | mg |
| hydroxypropylcellulose | 3.9 | mg |
| macrogol 6000 | 1.8 | mg |
| low-substituted | 13 | mg |
| hydroxypropylcellulose | | |
| magnesium stearate | 1.3 | mg |
| hypromellose | 3.829 | mg |
| titanium dioxide | 0.512 | mg |
| macrogol 6000 | 0.768 | mg |
| red ferric oxide | 0.011 | mg |
| yellow ferric oxide | 0.034 | mg |
| total | 135.154 | mg |

(1) Hydroxypropylcellulose (35.10 g) and macrogol 6000 (16.20 g) were dissolved in purified water (403.7 g) to give a binding solution. Amlodipine besylate (10.40 g), compound B (30.00 g), D-mannitol (250.6 g) and microcrystalline cellulose (39.00 g) were uniformly mixed in a fluid bed granulator (Lab-1, POWREX Co., Ltd.), and the mixture was granulated while spraying the binding solution, and then dried to give a granule. A part of the obtained granule was sieved with a sieve (16 mesh) to give a milled granule.
(2) To the obtained milled granule (289.3 g) were added low-substituted hydroxypropylcellulose (32.50 g) and magnesium stearate (3.250 g), and they were mixed in a polyethylene bag (4.9 L) to give a mixed granule.
(3) The mixed granule obtained above was tableted by a rotary tableting machine (VEL-5, Kikusui Seisakusho) with a diameter of 6.5 mm punch (tableting pressure 5 kN, weight per tablet: 130 mg) to give core tablets.
(4) Hypromellose (57.44 g) and macrogol 6000 (11.52 g) were dissolved in purified water (375.0 g) to give film coating solution I. Titanium dioxide (7.680 g), red ferric oxide (0.1650 g) and yellow ferric oxide (0.5100 g) were dispersed in purified water (330.0 g) to give a dispersion I. Film coating solution I, dispersion I and purified water (68.25 g) were mixed to give film coating solution II. A film coating was formed by uniformly spraying film coating solution II on the core tablets (120.0 g) in a film coating machine (DRC-200, POWREX Co., Ltd.) to give film-coated tablets of the composition shown in Table 15 (weight per tablet: 135.154 mg).

### Example 16

TABLE 16
Composition per preparation (135.154 mg)

| Compound B | 10 | mg |
|---|---|---|
| amlodipine besylate | 6.93 | mg |
| | (5 mg as amlodipine) | |
| D-mannitol | 80.07 | mg |
| microcrystalline cellulose | 13 | mg |
| hydroxypropylcellulose | 3.9 | mg |
| macrogol 6000 | 1.8 | mg |
| low-substituted | 13 | mg |
| hydroxypropylcellulose | | |
| magnesium stearate | 1.3 | mg |
| hypromellose | 3.829 | mg |
| titanium dioxide | 0.512 | mg |
| macrogol 6000 | 0.768 | mg |
| red ferric oxide | 0.011 | mg |
| yellow ferric oxide | 0.034 | mg |
| total | 135.154 | mg |

(1) Hydroxypropylcellulose (35.10 g) and macrogol 6000 (16.20 g) were dissolved in purified water (403.7 g) to give a binding solution. Amlodipine besylate (20.79 g), compound B (30.00 g), D-mannitol (240.2 g) and microcrystalline cellulose (39.00 g) were uniformly mixed in a fluid bed granulator (Lab-1, POWREX Co., Ltd.), and the mixture was granulated while spraying the binding solution, and then dried to give a granule. A part of the obtained granule was sieved with a sieve (16 mesh) to give a milled granule.
(2) To the obtained milled granule (289.3 g) were added low-substituted hydroxypropylcellulose (32.50 g) and magnesium stearate (3.250 g), and they were mixed in a polyethylene bag (4.9 L) to give a mixed granule.
(3) The mixed granule obtained above was tableted by a rotary tableting machine (VEL-5, Kikusui Seisakusho) with a diameter of 6.5 mm punch (tableting pressure 5 kN, weight per tablet: 130 mg) to give core tablets.
(4) Hypromellose (57.44 g) and macrogol 6000 (11.52 g) were dissolved in purified water (375.0 g) to give film coating solution I. Titanium dioxide (7.680 g), red ferric oxide (0.1650 g) and yellow ferric oxide (0.5100 g) were dispersed in purified water (330.0 g) to give a dispersion I. Film coating solution I, dispersion I and purified water (68.25 g) were mixed to give film coating solution II. A film coating was formed by uniformly spraying film coating solution II on the core tablets (120.0 g) in a film coating machine (DRC-200, POWREX Co., Ltd.) to give film-coated tablets of the composition shown in Table 16 (weight per tablet: 135.154 mg).

### Example 17

TABLE 17
Composition per preparation (135.119 mg)

| Compound B | 20 | mg |
|---|---|---|
| amlodipine besylate | 3.465 | mg |
| | (2.5 mg as amlodipine) | |
| D-mannitol | 73.535 | mg |
| microcrystalline cellulose | 13 | mg |
| hydroxypropylcellulose | 3.9 | mg |
| macrogol 6000 | 1.8 | mg |
| low-substituted | 13 | mg |
| hydroxypropylcellulose | | |
| magnesium stearate | 1.3 | mg |
| hypromellose | 3.829 | mg |
| titanium dioxide | 0.512 | mg |
| macrogol 6000 | 0.768 | mg |
| red ferric oxide | 0.01 | mg |
| total | 135.119 | mg |

(1) Hydroxypropylcellulose (35.10 g) and macrogol 6000 (16.20 g) were dissolved in purified water (403.7 g) to give a binding solution. Amlodipine besylate (10.40 g), compound B (60.00 g), D-mannitol (220.6 g) and microcrystalline cellulose (39.00 g) were uniformly mixed in a fluid bed granulator (Lab-1, POWREX Co., Ltd.), and the mixture was granulated while spraying the binding solution, and then dried to give a granule. A part of the obtained granule was sieved with a sieve (16 mesh) to give a milled granule.
(2) To the obtained milled granule (289.3 g) were added low-substituted hydroxypropylcellulose (32.50 g) and magnesium stearate (3.250 g), and they were mixed in a polyethylene bag (4.9 L) to give a mixed granule.
(3) The mixed granule obtained above was tableted by a rotary tableting machine (VEL-5, Kikusui Seisakusho) with a diameter of 6.5 mm punch (tableting pressure 5 kN, weight per tablet: 130 mg) to give core tablets.
(4) Hypromellose (57.44 g) and macrogol 6000 (11.52 g) were dissolved in purified water (375.0 g) to give film coating solution I. Titanium dioxide (7.680 g) and red ferric oxide (0.1500 g) were dispersed in purified water (330.0 g) to give a dispersion I. Film coating solution I, dispersion I and purified water (68.25 g) were mixed to give film coating solution II. A film coating was formed by uniformly spraying film coating solution II on the core tablets (120.0 g) in a film coating machine (DRC-200, POWREX Co., Ltd.) to give film-coated tablets of the composition shown in Table 17 (weight per tablet: 135.119 mg).

### Example 18

TABLE 18
Composition per preparation (135.119 mg)

| Compound B | 20 | mg |
|---|---|---|
| amlodipine besylate | 6.93 | mg |
| | (5 mg as amlodipine) | |
| D-mannitol | 70.07 | mg |
| microcrystalline cellulose | 13 | mg |
| hydroxypropylcellulose | 3.9 | mg |
| macrogol 6000 | 1.8 | mg |
| low-substituted | 13 | mg |
| hydroxypropylcellulose | | |
| magnesium stearate | 1.3 | mg |
| hypromellose | 3.829 | mg |
| titanium dioxide | 0.512 | mg |
| macrogol 6000 | 0.768 | mg |
| red ferric oxide | 0.01 | mg |
| total | 135.119 | mg |

(1) Hydroxypropylcellulose (35.10 g) and macrogol 6000 (16.20 g) were dissolved in purified water (403.7 g) to give a binding solution. Amlodipine besylate (20.79 g), compound B (60.00 g), D-mannitol (210.2 g) and microcrystalline cellulose (39.00 g) were uniformly mixed in a fluid bed granulator (Lab-1, POWREX Co., Ltd.), and the mixture was granulated while spraying the binding solution, and then dried to give a granule. A part of the obtained granule was sieved with a sieve (16 mesh) to give a milled granule.
(2) To the obtained milled granule (289.3 g) were added low-substituted hydroxypropylcellulose (32.50 g) and magnesium stearate (3.250 g), and they were mixed in a polyethylene bag (4.9 L) to give a mixed granule.
(3) The mixed granule obtained above was tableted by a rotary tableting machine (VEL-5, Kikusui Seisakusho) with a diameter of 6.5 mm punch (tableting pressure 5 kN, weight per tablet: 130 mg) to give core tablets.
(4) Hypromellose (57.44 g) and macrogol 6000 (11.52 g) were dissolved in purified water (375.0 g) to give film coating solution I. Titanium dioxide (7.680 g) and red ferric oxide (0.1500 g) were dispersed in purified water (330.0 g) to give a dispersion I. Film coating solution I, dispersion I and purified water (68.25 g) were mixed to give film coating solution II. A film coating was formed by uniformly spraying film coating solution II on the core tablets (120.0 g) in a film coating machine (DRC-200, POWREX Co., Ltd.) to give film-coated tablets of the composition shown in Table 18 (weight per tablet: 135.119 mg).

### Example 19

TABLE 19
Composition per preparation (135.155 mg)

| Compound B | 40 | mg |
|---|---|---|
| amlodipine besylate | 3.465 | mg |
| | (2.5 mg as amlodipine) | |
| D-mannitol | 53.535 | mg |
| microcrystalline cellulose | 13 | mg |
| hydroxypropylcellulose | 3.9 | mg |
| macrogol 6000 | 1.8 | mg |
| low-substituted | 13 | mg |
| hydroxypropylcellulose | | |
| magnesium stearate | 1.3 | mg |
| hypromellose | 3.829 | mg |
| titanium dioxide | 0.512 | mg |
| macrogol 6000 | 0.768 | mg |
| yellow ferric oxide | 0.046 | mg |
| total | 135.155 | mg |

(1) Hydroxypropylcellulose (35.10 g) and macrogol 6000 (16.20 g) were dissolved in purified water (403.7 g) to give a binding solution. Amlodipine besylate (10.40 g), compound B (120.0 g), D-mannitol (160.6 g) and microcrystalline cellulose (39.00 g) were uniformly mixed in a fluid bed granulator (Lab-1, POWREX Co., Ltd.), and the mixture was granulated while spraying the binding solution, and then dried to give a granule. A part of the obtained granule was sieved with a sieve (16 mesh) to give a milled granule.
(2) To the obtained milled granule (289.3 g) were added low-substituted hydroxypropylcellulose (32.50 g) and magnesium stearate (3.250 g), and they were mixed in a polyethylene bag (4.9 L) to give a mixed granule.
(3) The mixed granule obtained above was tableted by a rotary tableting machine (VEL-5, Kikusui Seisakusho) with a diameter of 6.5 mm punch (tableting pressure 5 kN, weight per tablet: 130 mg) to give core tablets.
(4) Hypromellose (57.44 g) and macrogol 6000 (11.52 g) were dissolved in purified water (375.0 g) to give film coating solution I. Titanium dioxide (7.680 g) and yellow ferric oxide (0.6900 g) were dispersed in purified water (330.0 g) to give a dispersion I. Film coating solution I, dispersion I and purified water (68.25 g) were mixed to give film coating solution II. A film coating was formed by uniformly spraying film coating solution II on the core tablets (120.0 g) in a film coating machine (DRC-200, POWREX Co., Ltd.) to give film-coated tablets of the composition shown in Table 19 (weight per tablet: 135.155 mg).

### Example 20

TABLE 20
Composition per preparation (135.155 mg)

| Compound B | 40 | mg |
|---|---|---|
| amlodipine besylate | 6.93 | mg |
| | (5 mg as amlodipine) | |
| D-mannitol | 50.07 | mg |
| microcrystalline cellulose | 13 | mg |
| hydroxypropylcellulose | 3.9 | mg |
| macrogol 6000 | 1.8 | mg |
| low-substituted | 13 | mg |
| hydroxypropylcellulose | | |
| magnesium stearate | 1.3 | mg |
| hypromellose | 3.829 | mg |
| titanium dioxide | 0.512 | mg |
| macrogol 6000 | 0.768 | mg |
| yellow ferric oxide | 0.046 | mg |
| total | 135.155 | mg |

(1) Hydroxypropylcellulose (35.10 g) and macrogol 6000 (16.20 g) were dissolved in purified water (403.7 g) to give a binding solution. Amlodipine besylate (20.79 g), compound B (120.0 g), D-mannitol (150.2 g) and microcrystalline cellulose (39.00 g) were uniformly mixed in a fluid bed granulator (Lab-1, POWREX Co., Ltd.), and the mixture was granulated while spraying the binding solution, and then dried to give a granule. A part of the obtained granule was sieved with a sieve (16 mesh) to give a milled granule.
(2) To the obtained milled granule (289.3 g) were added low-substituted hydroxypropylcellulose (32.50 g) and magnesium stearate (3.250 g), and they were mixed in a polyethylene bag (4.9 L) to give a mixed granule.
(3) The mixed granule obtained above was tableted by a rotary tableting machine (VEL-5, Kikusui Seisakusho) with a diameter of 7.0 mm punch (tableting pressure 5 kN, weight per tablet: 130 mg) to give core tablets.
(4) Hypromellose (57.44 g) and macrogol 6000 (11.52 g) were dissolved in purified water (375.0 g) to give film coating solution I. Titanium dioxide (7.680 g) and yellow ferric oxide (0.6900 g) were dispersed in purified water (330.0 g) to give a dispersion I. Film coating solution I, dispersion I and purified water (68.25 g) were mixed to give film coating solution II. A film coating was formed by uniformly spraying film coating solution II on the core tablets (120.0 g) in a film coating machine (DRC-200, POWREX Co., Ltd.) to give film-coated tablets of the composition shown in Table 20 (weight per tablet: 135.155 mg).

### Example 21

TABLE 21
Composition per preparation (260 mg)

| compound A | 8 | mg |
|---|---|---|
| amlodipine besylate | 6.93 | mg |
| | (5 mg as amlodipine) | |
| D-mannitol | 92.554 | mg |
| microcrystalline cellulose | 20 | mg |
| lactose hydrate | 89.384 | mg |
| corn starch | 20 | mg |
| hypromellose | 4 | mg |
| hydroxypropylcellulose | 4 | mg |
| macrogol 6000 | 2.6 | mg |
| croscarmellose sodium | 11.2 | mg |
| magnesium stearate | 1.3 | mg |
| red ferric oxide | 0.032 | mg |
| total | 260 | mg |

(1) Hypromellose (720.0 g) was dissolved in purified water (9000 g) to give binding solution I. Red ferric oxide (2.880 g) was dispersed in purified water (2880 g) to give dispersion I. Dispersion I and purified water (720.0 g) were mixed in binding solution I to give binding solution II. Amlodipine besylate (1249 g), D-mannitol (16660 g) and microcrystalline cellulose (3600 g) were uniformly mixed in a fluid bed granulator (FD-S2, POWREX Co., Ltd.), and the mixture was granulated while spraying binding solution II, and then dried to give a granule. A part of the obtained granule was milled with a 1.5 mmφ punching screen in a screening mill (P-3, Showa Kagakukikai Co., Ltd.) to give milled granule I.
(2) Hydroxypropylcellulose (720.0 g) and macrogol 6000 (468.0 g) were dissolved in purified water (9000 g) to give binding solution III. Red ferric oxide (2.880 g) was dispersed in purified water (2880 g) to give dispersion II. Dispersion II and purified water (720.0 g) were mixed in binding solution III to give binding solution IV. Compound A (1436 g), lactose hydrate (16090 g) and corn starch (3600 g) were uniformly mixed in a fluid bed granulator (FD-S2, POWREX Co., Ltd.), and the mixture was granulated while spraying binding solution IV, and then dried to give a granule. A part of the obtained granule was milled with a 1.5 mmφ punching screen in a screening mill (P-3, Showa Kagakukikai Co., Ltd.) to give milled granule II.
(3) To the obtained milled granule I (20380 g) and milled granule II (20460 g) were added croscarmellose sodium (1848 g) and magnesium stearate (214.5 g), and they were mixed in a tumbler mixer (TM20-0-0, Suchiro Kakoki co., Ltd.) to give a mixed granule.
(4) The mixed granule obtained above was tableted by a rotary tableting machine (AQUARIUS-36K, Kikusui Seisakusho) with a diameter of 8.5 mm punch (tableting pressure 10 kN, weight per tablet: 260 mg) to give plain tablets of the composition shown in Table 21.

### Example 22

TABLE 22
Composition per preparation (260 mg)

| compound A | 8 | mg |
|---|---|---|
| amlodipine besylate | 6.93 | mg |
| | (5 mg as amlodipine) | |
| D-mannitol | 92.554 | mg |
| microcrystalline cellulose | 20 | mg |
| lactose hydrate | 89.384 | mg |
| corn starch | 20 | mg |
| hypromellose | 4 | mg |
| hydroxypropylcellulose | 4 | mg |
| macrogol 6000 | 2.6 | mg |
| croscarmellose sodium | 5.6 | mg |
| carmellose calcium | 5.6 | mg |
| magnesium stearate | 1.3 | mg |
| red ferric oxide | 0.032 | mg |
| total | 260 | mg |

(1) Hypromellose (720.0 g) was dissolved in purified water (9000 g) to give binding solution I. Red ferric oxide (2.880 g) was dispersed in purified water (2880 g) to give dispersion I. Binding solution I was mixed with dispersion I and purified water (720.0 g) to give binding solution II. Amlodipine besylate (1249 g), D-mannitol (16660 g) and microcrystalline cellulose (3600 g) were uniformly mixed in a fluid bed granulator (FD-S2, POWREX Co., Ltd.), and the mixture was granulated while spraying binding solution II, and then dried to give a granule. A part of the obtained granule was milled with a 1.5 mmφ punching screen in a screening mill (P-3, Showa Kagakukikai Co., Ltd.) to give milled granule I.
(2) To the obtained milled granule I (20380 g) were added croscarmellose sodium (924.0 g) and magnesium stearate (148.5 g), and they were mixed in a tumbler mixer (TM-60, Showa Kagakukikai Co., Ltd.) to give mixed granule I.
(3) Hydroxypropylcellulose (720.0 g) and macrogol 6000 (468.0 g) were dissolved in purified water (9000 g) to give binding solution III. Red ferric oxide (2.880 g) was dispersed in purified water (2880 g) to give dispersion II. Dispersion II and purified water (720.0 g) were mixed in binding solution III to give binding solution IV. Compound A (1436 g), lactose hydrate (16090 g) and corn starch (3600 g) were uniformly mixed in a fluid bed granulator (FD-S2, POWREX Co., Ltd.), and the mixture was granulated while spraying binding solution IV, and then dried to give a granule. A part of the obtained granule was milled with a 1.5 mmφ punching screen in a screening mill (P-3, Showa Kagakukikai Co., Ltd.) to give milled granule II.
(4) To the obtained milled granule II (20460 g) were added carmellose calcium (924.0 g) and magnesium stearate (66.00 g), and they were mixed in a tumbler mixer (TM-60, Showa Kagakukikai Co., Ltd.) to give mixed granule II.
(5) Mixed granule I (130 mg) and mixed granule II (130 mg) obtained above were tableted by a rotary tableting machine (HT-CVX54LS-UW/C&3L, HATA IRON WORKS Co., Ltd.) with a diameter of 8.5 mm punch (tableting pressure 9 kN, weight per tablet: 260 mg) to give multi-layer plain tablets of the composition shown in Table 22.

### Example 23

TABLE 23
Composition per preparation (239 mg)

| Compound B | 40 | mg |
|---|---|---|
| lactose hydrate | 29.3 | mg |
| corn starch | 13 | mg |
| microcrystalline cellulose | 13 | mg |
| hydroxypropylcellulose | 4 | mg |
| macrogol 6000 | 4 | mg |
| low-substituted | 13 | mg |
| hydroxypropylcellulose | | |
| microcrystalline cellulose | 13 | mg |
| magnesium stearate | 0.7 | mg |
| amlodipine besylate | 6.93 | mg |
| | (5 mg as amlodipine) | |
| D-mannitol | 68.94 | mg |
| microcrystalline cellulose | 15.33 | mg |
| hydroxypropylcellulose | 3.1 | mg |
| carmellose calcium | 5 | mg |
| magnesium stearate | 0.7 | mg |
| premix I | 9 | mg |
| total | 239 | mg |

(1) Hydroxypropylcellulose (155.0 g) was dissolved in purified water (1395.0 g) to give binding solution I. Amlodipine besylate (346.7 g), D-mannitol (2447.0 g) and microcrystalline cellulose (766.7 g) were uniformly mixed in a fluid bed granulator (FD-5S, POWREX Co., Ltd.), and the mixture was granulated while spraying binding solution I, and then dried to give a granule of an amlodipine besylate layer. A part of the obtained granule was milled with a 1.5 mmφ punching screen in a screening mill (P-3, Showa Kagakukikai Co., Ltd.) to give a milled granule of the amlodipine besylate layer.
(2) Hydroxypropylcellulose (280.1 g) and macrogol 6000 (280.0 g) were dissolved in purified water (2520.2 g) to give binding solution II. Compound B (2808.4 g), lactose hydrate (2043.5 g), corn starch (910.3 g) and microcrystalline cellulose (910.2 g) were uniformly mixed in a fluid bed granulator (FD-5S, POWREX Co., Ltd.), and the mixture was granulated while spraying binding solution II, and then dried to give a granule of the compound B layer. A part of the obtained granule was milled with a 1.5 mmφ punching screen in a screening mill (P-3, Showa Kagakukikai Co., Ltd.) to give a milled granule of the compound B layer.
(3) To the milled granule of the obtained amlodipine besylate layer (3772.0 g) were added carmellose calcium (200.0 g) and magnesium stearate (28.000 g), and they were mixed in a tumbler mixer (TM-15, Suchiro Kakoki co., Ltd.) to give a mixed granule of the amlodipine besylate layer.
(4) To the milled granule of the obtained compound B layer (3616.1 g) were added low-substituted hydroxypropylcellulose (455.0 g), microcrystalline cellulose (455.1 g) and magnesium stearate (24.53 g), and they were mixed in a tumbler mixer (TM-15, Suchiro Kakoki co., Ltd.) to give a mixed granule of the compound B layer.
(5) The mixed granule of the amlodipine besylate layer and the mixed granule of the compound B layer obtained above were tableted by a rotary tableting machine (HT-X12SS-UW&2L, HATA IRON WORKS Co., Ltd.) with a diameter of 8.0 mm punch (tableting pressure 7 kN, weight per tablet: 230 mg (amlodipine besylate layer: 100 mg, compound B layer: 130 mg)) to give multi-layer core tablets.
(6) Premix I (252.0 g) was dissolved in purified water (2268.0 g) to give a film coating solution. A film coating was formed by uniformly spraying the film coating solution on the core tablets (3120.0 g) in a film coating machine (DRC-500, POWREX Co., Ltd.) to give film-coated tablets of the composition shown in Table 23 (weight per tablet: 239 mg). Here, the premix I is a premixed powder. The composition of the premix I is shown in Table 23a.

TABLE 23a
Composition of premix I

| | ratio by weight |
|---|---|
| hypromellose | 9.0 |
| macrogol 6000 | 2.0 |
| titanium dioxide | 1.0 |
| yellow ferric oxide | 0.2 |

### Example 24

TABLE 24
Composition per preparation (130 mg)

| compound A | 8 | mg |
|---|---|---|
| amlodipine besylate | 6.93 | mg |
| | (5 mg as amlodipine) | |
| D-mannitol | 82.666 | mg |
| microcrystalline cellulose | 20 | mg |
| hydroxypropylcellulose | 4 | mg |
| macrogol 4000 | 1.8 | mg |
| croscarmellose sodium | 5.6 | mg |
| magnesium stearate | 0.9 | mg |
| red ferric oxide | 0.104 | mg |
| total | 130 | mg |

(1) Hydroxypropylcellulose (80.00 g) and macrogol 4000 (36.00 g) were dissolved in purified water (1100 g) to give binding solution I. Red ferric oxide (2.080 g) was dispersed in purified water (200.1 g) to give dispersion I. Binding solution I was mixed with dispersion I and purified water (60.00 g) to give binding solution II. Amlodipine besylate (24.95 g), compound A (28.80 g), D-mannitol (297.6 g) and microcrystalline cellulose (72.00 g) were uniformly mixed in a fluid bed granulator (Lab-1, POWREX Co., Ltd.), and the mixture was granulated while spraying binding solution II (266.1 g), and then dried to give a granule. A part of the obtained granule was sieved with a sieve (16 mesh) to give a milled granule.
(2) To the obtained milled granule (370.5 g) were added croscarmellose sodium (16.81 g) and magnesium stearate (2.700 g), and they were mixed in a polyethylene bag (4.9 L) to give a mixed granule.
(3) The mixed granule obtained above was tableted by a rotary tableting machine (Correct 19K, Kikusui Seisakusho) with a long diameter of 8.5 mm and short diameter of 5.0 mm punch (tableting pressure 8.5 kN, weight per tablet: 130 mg) to give plain tablets of the composition shown in Table 24.

### Example 25

TABLE 25
Composition per preparation (130 mg)

| compound A | 8 | mg |
|---|---|---|
| amlodipine besylate | 6.93 | mg |
| | (5 mg as amlodipine) | |
| D-mannitol | 82.666 | mg |
| microcrystalline cellulose | 20 | mg |
| hydroxypropylcellulose | 4 | mg |
| macrogol 10000 | 1.8 | mg |
| croscarmellose sodium | 5.6 | mg |
| magnesium stearate | 0.9 | mg |
| red ferric oxide | 0.104 | mg |
| total | 130 | mg |

(1) Hydroxypropylcellulose (80.00 g) and macrogol 10000 (36.00 g) were dissolved in purified water (1100 g) to give binding solution I. Red ferric oxide (2.080 g) was dispersed in purified water (200.1 g) to give dispersion I. Binding solution I was mixed with dispersion I and purified water (60.10 g) to give binding solution II. Amlodipine besylate (24.95 g), compound A (28.80 g), D-mannitol (297.6 g) and microcrystalline cellulose (72.00 g) were uniformly mixed in a fluid bed granulator (Lab-1, POWREX Co., Ltd.), and the mixture was granulated while spraying binding solution II (266.1 g), and then dried to give a granule. A part of the obtained granule was sieved with a sieve (16 mesh) to give a milled granule.
(2) To the obtained milled granule (370.5 g) were added croscarmellose sodium (16.80 g) and magnesium stearate (2.710 g), and they were mixed in a polyethylene bag (4.9 L) to give a mixed granule.
(3) The mixed granule obtained above was tableted by a rotary tableting machine (Correct 19K, Kikusui Seisakusho) with a long diameter of 8.5 mm and short diameter of 5.0 mm punch (tableting pressure 8.5 kN, weight per tablet: 130 mg) to give plain tablets of the composition shown in Table 25.

### Comparative Example 1

TABLE 26
Composition per preparation (130 mg)

| compound A | 8 | mg |
|---|---|---|
| amlodipine besylate | 6.93 | mg |
| | (5 mg as amlodipine) | |
| lactose hydrate | 82.454 | mg |
| corn starch | 20 | mg |
| hydroxypropylcellulose | 4 | mg |
| macrogol 6000 | 2.6 | mg |
| carmellose calcium | 5.6 | mg |
| magnesium stearate | 0.4 | mg |
| red ferric oxide | 0.016 | mg |
| total | 130 | mg |

(1) Hydroxypropylcellulose (720.0 g) and macrogol 6000 (468.0 g) were dissolved in purified water (9000 g) to give binding solution I. Red ferric oxide (2.880 g) was dispersed in purified water (2880 g) to give dispersion I. Binding solution I was mixed with dispersion I and purified water (720.0 g) to give binding solution II. Amlodipine besylate (1253 g), compound A (1449 g), lactose hydrate (14830 g) and corn starch (3600 g) were uniformly mixed in a fluid bed granulator (FD-S2, POWREX Co., Ltd.), and the mixture was granulated while spraying binding solution II, and then dried to give a granule. A part of the obtained granule was milled with a 1.5 mmφ punching screen in a screening mill (P-3, Showa Kagakukikai Co., Ltd.) to give a milled granule.
(2) To the obtained milled granule (40920 g (2 batches)) were added carmellose calcium (1848 g) and magnesium stearate (132.0 g), and they were mixed in a tumbler mixer (TM20-0-0, Suchiro Kakoki co., Ltd.) to give a mixed granule.
(3) The mixed granule obtained above was tableted by a rotary tableting machine (AQUARIUS-36K, Kikusui Seisakusho) with a diameter of 7.0 mm punch (tableting pressure 9 kN, weight per tablet: 130 mg) to give plain tablets of the composition shown in Table 26.

### Experimental Example 1

### Dissolution Test 1

The dissolution property of the active ingredient (compound A) from the plain tablets obtained in Examples 1 to 11, 24, 25 and Comparative Example 1 was evaluated by a dissolution test (1.0 (w/w) % polysorbate 20 solution, 900 mL, 37° C., Paddle Method, number of rotation 50 rpm). The dissolution test was performed according to the Japanese Pharmacopoeia, 15th Edition, Dissolution Test, Apparatus 2 (Paddle Method). The results are shown in Table 27. Table 27 shows average values, maximum values and minimum values of the dissolution rate after 15 min from the start of the dissolution. Examples 1, 4, 5, 8 to 11, 24, 25 and Comparative Example 1 show average values, maximum values and minimum values of the dissolution rates of 6 tablets, and Examples 2, 3, 6 and 7 show average values, maximum values and minimum values of the dissolution rates of 12 tablets.

TABLE 27

| | dissolution rate (%) after 15 min | | |
|---|---|---|---|
| | average | maximum | minimum |
| sample | value | value | value |
| Comparative | 43 | 47 | 39 |
| Example 1 | | | |
| Example 1 | 56 | 60 | 51 |
| Example 2 | 78 | 84 | 64 |
| Example 3 | 75 | 81 | 70 |
| Example 4 | 68 | 75 | 61 |
| Example 5 | 67 | 72 | 64 |
| Example 6 | 74 | 82 | 65 |
| Example 7 | 74 | 79 | 69 |
| Example 8 | 75 | 80 | 71 |
| Example 9 | 74 | 78 | 71 |
| Example 10 | 62 | 65 | 57 |
| Example 11 | 49 | 56 | 39 |
| Example 24 | 75 | 83 | 68 |
| Example 25 | 82 | 85 | 75 |

As shown in Table 27, all of the plain tablets of Examples 1 to 11, 24, 25 containing sugar alcohol as an excipient showed good dissolution property of compound A in comparison to the sugar alcohol-free plain tablet of Comparative Example 1.

### Experimental Example 2

### Dissolution Test 2

The dissolution property of active ingredient (compound B) from the film-coated tablet obtained in Example 20 was evaluated by a dissolution test (the Japanese Pharmacopoeia, 15th Edition, 2nd fluid for dissolution test, 900 mL, 37° C., Paddle Method, number of rotation 50 rpm). The dissolution test was performed according to the Japanese Pharmacopoeia, 15th Edition, Dissolution Test, Apparatus 2 (Paddle Method). The results are shown in Table 28. Table 28 shows average values, maximum values and minimum values of the dissolution rate of 6 tablets at each time point after 5 to 60 min from the start of the dissolution.

TABLE 28

| | dissolution rate (%) | | |
|---|---|---|---|
| measurement | average | maximum | minimum |
| time (min later) | value | value | value |
| 5 | 34 | 30 | 37 |
| 10 | 77 | 74 | 81 |
| 15 | 90 | 88 | 92 |
| 20 | 92 | 90 | 95 |
| 25 | 94 | 92 | 96 |
| 30 | 95 | 93 | 96 |
| 45 | 96 | 94 | 99 |
| 60 | 97 | 95 | 101 |

As shown in Table 28, the film-coated tablet of Example 20 showed good dissolution property of compound B.

### Experimental Example 3

### Stability Test

The plain tablets of Examples 1, 4 and 5 were preserved under conditions of sealed glass bottle and 25° C./60% RH for 12 months, the mass of analogues derived from compound A or amlodipine besylate was measured, and the preservation stability was evaluated based thereon. The results are shown in Table 29. The values in the Table 29 show the rates (%) of the total analogues derived from compound A or amlodipine besylate when the content of compound A or amlodipine besylate (compound A; 8 mg or 4 mg/amlodipine besylate; 6.93 mg or 3.47 mg) is 100%.

TABLE 29

| | | | total analogue |
|---|---|---|---|
| | | total analogue | derived from |
| | | derived from | amlodipine |
| Sample | conditions | compound A | besylate |
| Example 1 | when experiment was | 0.21% | 0.12% |
| | started | | |
| | 25° C./60% RH 12 months | 0.86% | 0.18% |
| | reserved product | | |
| Example 4 | when experiment was | 0.49% | 0.12% |
| | started | | |
| | 25° C./60% RH 12 months | 1.21% | 0.21% |
| | reserved product | | |
| Example 5 | when experiment was | 0.48% | 0.18% |
| | started | | |
| | 25° C./60% RH 12 months | 1.12% | 0.29% |
| | reserved product | | |

As shown in Table 29, the plain tablets of Examples 1, 4 and 5 showed good preservation stability.

### INDUSTRIAL APPLICABILITY

The present invention provides a solid preparation comprising a compound represented by the formula (I) or a salt thereof, sugar alcohol, and a calcium antagonist, which appropriately controls dissolution of the compound represented by the formula (I) or a salt thereof and the calcium antagonist from the solid preparation in the gastrointestinal tract, and maintains good stability thereof in the solid preparation.

This application is based on patent application Nos. 2009-111381 and 2010-68625 filed in Japan, the contents of which are incorporated in full herein by this reference.

1. A solid preparation comprising (i) a compound represented by the formula (I): 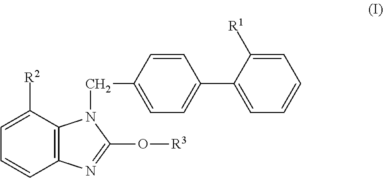
wherein R¹ is a monocyclic nitrogen-containing heterocyclic group having a deprotonatable hydrogen atom, R² is an optionally esterified carboxyl group, and R³ is an optionally substituted lower alkyl group, or a salt thereof, (ii) a sugar alcohol, and (iii) a calcium antagonist.

2. The solid preparation according to claim 1, wherein the compound represented by the formula (I) or a salt thereof is 1-(cyclohexyloxycarbonyloxy)ethyl 2-ethoxy-1-[[2′-(1H-tetrazol-5-yl)biphenyl-4-yl]methyl]benzimidazole-7-carboxylate, 2-ethoxy-1-[[2′-(1H-tetrazol-5-yl)biphenyl-4-yl]methyl]-1H-benzimidazole-7-carboxylic acid, 2-ethoxy-1-[[2′-(4,5-dihydro-5-oxo-1,2,4-oxadiazol-3-yl)biphenyl-4-yl]methyl]-1H-benzimidazole-7-carboxylic acid or a salt thereof.

3. The solid preparation according to claim 1, wherein the compound represented by the formula (I) or a salt thereof is 1-(cyclohexyloxycarbonyloxy)ethyl 2-ethoxy-1-[[2′-(1H-tetrazol-5-yl)biphenyl-4-yl]methyl]benzimidazole-7-carboxylate or a salt thereof.

4. The solid preparation according to claim 1, wherein the compound represented by the formula (I) or a salt thereof is 2-ethoxy-1-[[2′-(4,5-dihydro-5-oxo-1,2,4-oxadiazol-3-yl)biphenyl-4-yl]methyl]-1H-benzimidazole-7-carboxylic acid or a salt thereof.

5. The solid preparation according to claim 1, wherein the sugar alcohol is mannitol, sorbitol or erythritol.

6. The solid preparation according to claim 1, wherein the sugar alcohol is mannitol.

7. The solid preparation according to claim 1, wherein the calcium antagonist is azelnidipine, amlodipine, aranidipine, efonidipine, cilnidipine, nicardipine, nisoldipine, nitrendipine, nifedipine, nilvadipine, barnidipine, felodipine, benidipine, manidipine or a salt thereof.

8. The solid preparation according to claim 1, wherein the calcium antagonist is amlodipine or a salt thereof.

9. The solid preparation according to claim 1, further comprising a polyethylene glycol.

10. The solid preparation according to claim 9, wherein the polyethylene glycol has a molecular weight of 1,000 to 10,000.

11. A solid preparation comprising (i) 1-(cyclohexyloxycarbonyloxy)ethyl 2-ethoxy-1-[[2′-(1H-tetrazol-5-yl)biphenyl-4-yl]methyl]benzimidazole-7-carboxylate or a salt thereof, (ii) mannitol, and (iii) amlodipine or a salt thereof.

12. A solid preparation comprising (i) 2-ethoxy-1-[[2′-(4,5-dihydro-5-oxo-1,2,4-oxadiazol-3-yl)biphenyl-4-yl]methyl]-1H-benzimidazole-7-carboxylic acid or a salt thereof, (ii) mannitol, and (iii) amlodipine or a salt thereof.
